(19) United States
(12) Patent Application Publication
(10) Pub. No.: US 2023/0321196 A1
(43) Pub. Date: Oct. 12, 2023

(54) Medicine for Preventing or Treating Symptom or Disorder in Subject Affected by Viral Infection

(71) Applicant: University of Miyazaki, Miyazaki (JP)

(72) Inventors: Kazuo Kitamura, Miyazaki-shi, Miyazaki (JP); Toshihiro Kita, Miyazaki-shi, Miyazaki (JP)

(73) Assignee: University of Miyazaki, Miyazaki (JP)

(21) Appl. No.: 18/025,266

(22) PCT Filed: Sep. 8, 2021
(86) PCT No.: PCT/JP2021/032970

§ 371 Date: Mar. 23, 2023

(30) Foreign Application Priority Data
Sep. 9, 2020 (JP) 2020-151099

(51) Int. Cl. A61K 38/22; A61P 31/16; A61K 9/00

(52) U.S. Cl. CPC A61K 38/22; A61P 31/16; A61K 9/0019

(57) ABSTRACT

The present invention provides a means for preventing and/or treating a symptom of a viral infection in a subject affected by the viral infection without inducing any undesired serious side effect by optimizing the application method and the dose of a medicine containing AM or a derivative thereof as an active ingredient. One aspect of the present invention relates to a medicine for preventing or treating a symptom or disorder in a subject affected by a viral infection, the medicine containing adrenomedullin or a derivative thereof as an active ingredient.

### TECHNICAL FIELD

The present invention relates to a medicament for preventing or treating a symptom or disorder in a subject affected by a viral infection. In particular, an aspect of the present invention relates to a medicament for preventing or treating a symptom or disorder in a subject affected by a viral infection, which comprises adrenomedullin or a derivative thereof as an active ingredient.

### BACKGROUND ART

An infection due to novel coronavirus (hereinafter, also described as “SARS-CoV-2”) (hereinafter, this infection is also described as “COVID-19”) has been prevailing in the world since early 2020. COVID-19 is said to cause exacerbation to a certain percentage of patients. Hence, lifesaving for critically ill patients is a very important issue in treatment for COVID-19 patients. The mortality of COVID-19 is high in patients with exacerbated pneumonia. Moreover, situations beyond the capacities of conventional antiviral drugs currently under research have been generated.

In addition to SARS-CoV-2, various viruses including influenzavirus and severe acute respiratory syndrome (hereinafter, also described as “SARS”) coronavirus have been causing pandemics in recent years. These virus infections have killed many people by causing systemic inflammation, which leads to organ dysfunctions.

Adrenomedullin (hereinafter, also described as “AM”) is a bioactive peptide which was isolated and identified from pheochromocytoma in 1993 (Non Patent Literature 1). At the beginning of the discovery, AM was found to exert a strong vasodilatory hypotensive effect. For example, Patent Literature 1 describes a peptide having a blood pressure-lowering effect that comprises the amino acid sequence of human AM.

Subsequent studies revealed that AM exerts diverse pharmacological effects such as a cardiovascular protective effect, an anti-inflammatory effect, an angiogenic effect, a tissue repair promoting effect, and an organ protective effect. In an effort to apply the pharmacological effects of AM to treatment of disease, administration of AM to patients with different disease has been attempted. AM is expected to be useful as a therapeutic agent for inflammatory bowel disease, pulmonary hypertension, or a peripheral vascular disease, among others.

For example, Patent Literature 2 describes an agent for preventing or treating nonbacterial inflammatory bowel disease wherein the agent comprises, as an active ingredient, adrenomedullin or a derivative thereof that has an activity to suppress nonbacterial inflammation, or a salt thereof that has an activity to suppress nonbacterial inflammation.

Patent Literature 3 describes a method for preventing or treating an inflammatory bowel disease for which the use of a steroid formulation, an immunosuppressant, or a biological formulation is difficult or insufficiently effective in a patient in need of prevention or treatment of the inflammatory bowel disease, the method comprising administering an effective amount of adrenomedullin, a derivative thereof having an activity of suppressing inflammation, or a salt of the adrenomedullin or the derivative having an activity of suppressing inflammation, to the patient.

### CITATION LIST

### Patent Literature

Patent Literature 1: JP Patent No. 2774769
Patent Literature 2: JP Patent No. 4830093
Patent Literature 3: JP Patent No. 5954736

### Non Patent Literature

Non Patent Literature 1: Kitamura K, Kangawa K, Kawamoto M, Ichiki Y, Nakamura S, Matsuo H, Eto T. Adrenomedullin: a novel hypotensive peptide isolated from human pheochromocytoma. Biochem Biophys Res Commun, 30 Apr. 1993, Volume 192, Issue 2, pp. 553-560

### SUMMARY OF INVENTION

### Technical Problem

Currently, many viruses, including SARS-CoV-2 and influenzavirus, are known as viruses that cause viral infections. For these viruses, except a few viruses such as influenzavirus and herpesvirus, no effective therapeutic agent has been established yet. Viral infections caused by influenzavirus, varicella-zoster virus, and measles virus are each expected to be prevented by vaccination against the corresponding virus. However, even influenzavirus affects and kills many people every year in the world. Also in Japan, thousands of people die of the viral infection caused by influenzavirus every year. Therefore, new therapeutic means against viral infections that can be exacerbated has been demanded.

Since the cardiovascular protective effect, anti-inflammatory effect, angiogenic effect, tissue repair promoting effect, organ protective effect, or the like, of AM are known, as described above, the preventive and/or therapeutic effects of AM against symptoms or disorders, such as organ dysfunctions, in patients with viral infections were considered promising. However, there have been some problems in administration of AM to patients with viral infections.

AM has a strong vasodilatory hypotensive effect, as described above. Thus, from the viewpoint of management of blood pressure, it has been needed for long-term administration of AM to avoid administration at night, in which reduction in blood pressure is expected. In administering AM to patients with viral infections, excessively high rates of administration or excessive doses may cause reduction in blood pressure due to the excessive vasodilatory effect of AM, leading to a shock state.

Therefore, an object of the present invention is to provide means to prevent and/or treat a symptom due to a viral infection in a subject with the viral infection, without causing any unwanted serious side effect, by optimizing a dosage and administration of a medicament comprising AM or a derivative thereof as an active ingredient.

### Solution to Problem

The present inventors conducted various investigations of means to solve the problems described above. The present inventors have found that by administering a specific dose of AM to a patient with viral pneumonia, one of viral infections, according to a specific administration regimen, symptoms or disorders due to viral pneumonia can be prevented and/or treated without causing any unwanted serious side effect. The present inventors have achieved the invention based on the finding described above.

That is to say, the invention includes the following aspects and embodiments.

(1) A medicament for preventing or treating a symptom or disorder in a subject affected by a viral infection, or a subject affected by a sequela after cure of a viral infection, which comprises adrenomedullin or a derivative thereof as an active ingredient.

(2) The medicament according to the embodiment (1) for preventing or treating a symptom or disorder in a subject affected by a viral infection.

(3) The medicament according to the embodiment (1) or (2), further comprising one or more pharmaceutically acceptable carriers.

(4) The medicament according to any of the embodiments (1) to (3), wherein the adrenomedullin or a derivative thereof is a peptide or compound or a pharmaceutically acceptable salt thereof, or a pharmaceutically acceptable solvate thereof, the peptide or compound being selected from the group consisting of:
(i) a peptide consisting of an amino acid sequence of adrenomedullin,
(ii) a peptide consisting of an amino acid sequence of adrenomedullin and having a disulfide bond formed by two cysteine residues in the amino acid sequence,
(iii) a peptide wherein the disulfide bond of the peptide of (ii) is substituted with an ethylene group,
(iv) a peptide wherein 1 to 15 amino acid residues of any of the peptides of (i) to (iii) are deleted, substituted, or added,
(v) a peptide wherein any of the peptides of (i) to (iv) is amidated at the C-terminus thereof, (vi) a peptide wherein any of the peptides of (i) to (iv) has a glycine residue added to the C-terminus thereof, and
(vii) a compound represented by formula (A-I):

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (i) to (vi),
provided that modifying group A is bound to the N-terminal amino group of peptide group B with or without intervening linking group L.

(5) The medicament according to the embodiment (4), wherein the adrenomedullin or a derivative thereof is a peptide or compound or a pharmaceutically acceptable salt thereof, or a pharmaceutically acceptable solvate thereof, the peptide or compound being selected from the group consisting of:
(i) a peptide consisting of an amino acid sequence of adrenomedullin,
(ii) a peptide consisting of an amino acid sequence of adrenomedullin and having a disulfide bond formed by two cysteine residues in the amino acid sequence,
(v) a peptide wherein the peptide of (i) or (ii) is amidated at the C-terminus thereof,
(vi) a peptide wherein the peptide of (i) or (ii) has a glycine residue added to the C-terminus thereof, and
(vii) a compound represented by formula (A-I):

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (i), (ii), (v), and (vi),
provided that modifying group A is bound to the N-terminal amino group of peptide group B with or without intervening linking group L.

(6) The medicament according to any of the embodiments (1) to (3), wherein the adrenomedullin or a derivative thereof is a peptide or compound or a pharmaceutically acceptable salt thereof, or a pharmaceutically acceptable solvate thereof, the peptide or compound being selected from the group consisting of:
(a) a peptide consisting of an amino acid sequence of SEQ ID NO: 1, or a peptide consisting of an amino acid sequence of SEQ ID NO: 1, and having a disulfide bond formed by a cysteine residue at position 16 and a cysteine residue at position 21;
(b) a peptide consisting of an amino acid sequence of SEQ ID NO: 4, or a peptide consisting of an amino acid sequence of SEQ ID NO: 4, and having a disulfide bond formed by a cysteine residue at position 16 and a cysteine residue at position 21;
(c) a peptide consisting of an amino acid sequence of SEQ ID NO: 6, or a peptide consisting of an amino acid sequence of SEQ ID NO: 6, and having a disulfide bond formed by a cysteine residue at position 16 and a cysteine residue at position 21;
(d) a peptide consisting of an amino acid sequence of SEQ ID NO: 8, or a peptide consisting of an amino acid sequence of SEQ ID NO: 8, and having a disulfide bond formed by a cysteine residue at position 16 and a cysteine residue at position 21;
(e) a peptide consisting of an amino acid sequence of SEQ ID NO: 10, or a peptide consisting of an amino acid sequence of SEQ ID NO: 10, and having a disulfide bond formed by a cysteine residue at position 14 and a cysteine residue at position 19;
(f) a peptide consisting of an amino acid sequence of SEQ ID NO: 12, or a peptide consisting of an amino acid sequence of SEQ ID NO: 12, and having a disulfide bond formed by a cysteine residue at position 14 and a cysteine residue at position 19;
(g) a peptide wherein the disulfide bond of any of the peptides of (a) to (f) is substituted with an ethylene group;
(h) a peptide wherein 1 to 15 amino acid residues of any of the peptides of (a) to (g) are deleted, substituted, or added;
(i) a peptide wherein any of the peptides of (a) to (h) is amidated at the C-terminus thereof;
(j) a peptide wherein any of the peptides of (a) to (h) has a glycine residue added to the C-terminus thereof; and
(k) a compound represented by formula (A-I):

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (a) to (j),
provided that modifying group A is bound to the N-terminal amino group of peptide group B with or without intervening linking group L.

(7) The medicament according to the embodiment (6), wherein the adrenomedullin or a derivative thereof is a peptide or compound or a pharmaceutically acceptable salt thereof, or a pharmaceutically acceptable solvate thereof, the peptide or compound being selected from the group consisting of:
(a) a peptide consisting of an amino acid sequence of SEQ ID NO: 1, or a peptide consisting of an amino acid sequence of SEQ ID NO: 1, and having a disulfide bond formed by a cysteine residue at position 16 and a cysteine residue at position 21;
(b) a peptide consisting of an amino acid sequence of SEQ ID NO: 4, or a peptide consisting of an amino acid sequence of SEQ ID NO: 4, and having a disulfide bond formed by a cysteine residue at position 16 and a cysteine residue at position 21;
(c) a peptide consisting of an amino acid sequence of SEQ ID NO: 6, or a peptide consisting of an amino acid sequence of SEQ ID NO: 6, and having a disulfide bond formed by a cysteine residue at position 16 and a cysteine residue at position 21;
(d) a peptide consisting of an amino acid sequence of SEQ ID NO: 8, or a peptide consisting of an amino acid sequence of SEQ ID NO: 8, and having a disulfide bond formed by a cysteine residue at position 16 and a cysteine residue at position 21;
(e) a peptide consisting of an amino acid sequence of SEQ ID NO: 10, or a peptide consisting of an amino acid sequence of SEQ ID NO: 10, and having a disulfide bond formed by a cysteine residue at position 14 and a cysteine residue at position 19;
(f) a peptide consisting of an amino acid sequence of SEQ ID NO: 12, or a peptide consisting of an amino acid sequence of SEQ ID NO: 12, and having a disulfide bond formed by a cysteine residue at position 14 and a cysteine residue at position 19;
(i) a peptide wherein any of the peptide of (a) to (f) is amidated at the C-terminus thereof; (j) a peptide wherein any of the peptides of (a) to (f) has a glycine residue added to the C-terminus thereof; and
(k) a compound represented by formula (A-I):

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (a) to (f), (i), and (j),
provided that modifying group A is bound to the N-terminal amino group of peptide group B with or without intervening linking group L.

(8) The medicament according to any of the embodiments (1) to (4), wherein the peptide of (iv) in the adrenomedullin or a derivative thereof is a peptide wherein one to three amino acid residues of a peptide consisting of an amino acid sequence of SEQ ID NO: 14 are substituted or deleted.

(9) The medicament according to any of the embodiments (4) to (8), wherein modifying group A in formula (A-I) is a modifying group selected from the group consisting of an organic group comprising a C₄-C₃₀ alkyl group, a C₄-C₃₀ alkenyl group or a polyethylene glycol group, an Fc region of an immunoglobulin, and serum albumin.

(10) The medicament according to any of the embodiments (4) to (9), wherein, in formula (A-I),
A is an organic group comprising a polyethylene glycol group,
L is a divalent linking group represented by formula (z):

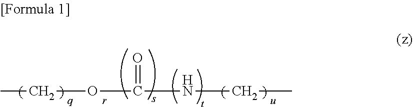

wherein
q and u are the same or different, and each represent an integer of 0 to 6, and
r, s, and t are the same or different, and each represent an integer of 0 or 1, and
n is the integer 1.

(11) The medicament according to the embodiment (10), wherein the organic group comprising a polyethylene glycol group is a group represented by any of formulas (13) to (6):

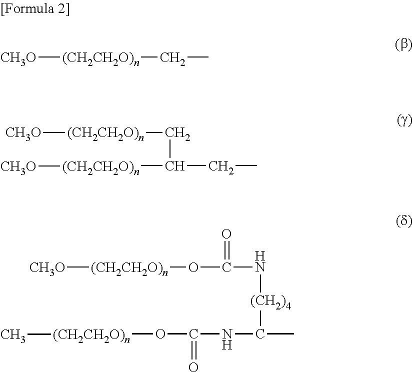

and,
having a weight-average molecular weight ranging from 20 to 80 kDa.

(12) The medicament according to any of the embodiments (1) to (11), wherein the adrenomedullin or a derivative thereof is adrenomedullin or a derivative thereof with adrenomedullin activity.

(13) The medicament according to any of the embodiments (1) to (12), wherein the viral infection is viral pneumonia, viral myocarditis, viral encephalitis, viral hemorrhagic fever, viral nephropathy, viral gastroenteritis, viral vasculitis, viral stomatitis, viral keratitis, or viral neuritis.

(14) The medicament according to any of the embodiments (1) to (12), wherein the viral infection in the subject affected by the viral infection is caused by one or more viruses selected from the group consisting of novel coronavirus (SARS-CoV-2), severe acute respiratory syndrome (SARS) coronavirus, Middle East respiratory syndrome (MERS) coronavirus, conventional human coronavirus (229E, NL63, OC43, and HKU1), influenzavirus, dengue virus, RS virus, adenovirus, varicella-zoster virus, herpes simplex virus, measles virus, parainfluenza virus, enterovirus, rhinovirus, and human metapneumovirus.

(15) The medicament according to any of the embodiments (1) and (3) to (14), wherein the sequela remaining after cure of the viral infection is dyspnea or shortness of breath remaining after cure of viral pneumonia.

(16) The medicament according to any of the embodiments (1) to (15), wherein the adrenomedullin or a derivative thereof as an active ingredient is applied through intravenous administration in a continuous manner.

(17) The medicament according to the embodiment (16), wherein the intravenous administration is performed in a continuous manner at a rate ranging from 1.0 to 100.0 ng active ingredient/kg body weight/min.

(18) The medicament according to the embodiment (16) or (17), wherein the intravenous administration is performed with continuous administration for 72 hours followed by intermittent administration for 8 hours per day.

(19) The medicament according to any of the embodiments (16) to (18), wherein the intravenous administration is performed for 3 to 10 days after initiation of administration.

(20) The medicament according to any of the embodiments (16) to (19), wherein the intravenous administration is performed on the basis of an administration regimen such that the intravenous administration is performed with continuous administration for 72 hours followed by intermittent administration for 8 hours per day for 3 to 10 days after initiation of administration.

(21) A pharmaceutical composition for preventing or treating a symptom or disorder in a subject affected by a viral infection, or a subject affected by a sequela after cure of a viral infection, which comprises: the adrenomedullin or a derivative thereof defined in any of the embodiments (1) to (20), or the medicament according to any of the embodiments (1) to (20); and one or more pharmaceutically acceptable carriers.

(22) The pharmaceutical composition according to the embodiment (21), wherein the viral infection is caused by one or more viruses selected from the group consisting of novel coronavirus (SARS-CoV-2), severe acute respiratory syndrome (SARS) coronavirus, Middle East respiratory syndrome (MERS) coronavirus, conventional human coronavirus (229E, NL63, OC43, and HKU1), influenzavirus, dengue virus, RS virus, adenovirus, varicella-zoster virus, herpes simplex virus, measles virus, parainfluenza virus, enterovirus, rhinovirus, and human metapneumovirus.

(23) The pharmaceutical composition according to the embodiment (21) or (22), wherein the viral infection is viral pneumonia, viral myocarditis, viral encephalitis, viral hemorrhagic fever, viral nephropathy, viral gastroenteritis, viral vasculitis, viral stomatitis, viral keratitis, or viral neuritis.

(24) A method for preventing or treating a symptom or disorder due to a viral infection or a sequela after cure of a viral infection, which comprises administering an effective amount of the adrenomedullin or a derivative thereof defined in any of the embodiments (1) to (20), or the medicament according to any of the embodiments (1) to (20), to a subject affected by the viral infection or a subject affected by the sequela after cure of the viral infection.

(25) The method according the embodiment (24), wherein the viral infection is caused by one or more viruses selected from the group consisting of novel coronavirus (SARS-CoV-2), severe acute respiratory syndrome (SARS) coronavirus, Middle East respiratory syndrome (MERS) coronavirus, conventional human coronavirus (229E, NL63, OC43, and HKU1), influenzavirus, dengue virus, RS virus, adenovirus, varicella-zoster virus, herpes simplex virus, measles virus, parainfluenza virus, enterovirus, rhinovirus, and human metapneumovirus.

(26) The method according to the embodiment (24) or (25), wherein the viral infection is viral pneumonia, viral myocarditis, viral encephalitis, viral hemorrhagic fever, viral nephropathy, viral gastroenteritis, viral vasculitis, viral stomatitis, viral keratitis, or viral neuritis.

(27) The adrenomedullin or a derivative thereof defined in any of the embodiments (1) to (20), or the medicament according to any of the embodiments (1) to (20), for use in the prevention or treatment of a symptom or disorder due to a viral infection, and a sequela after cure of a viral infection.

(28) The adrenomedullin or a derivative thereof, or an antiviral agent for use according to the embodiment (27), wherein the viral infection is caused by one or more viruses selected from the group consisting of novel coronavirus (SARS-CoV-2), severe acute respiratory syndrome (SARS) coronavirus, Middle East respiratory syndrome (MERS) coronavirus, conventional human coronavirus (229E, NL63, OC43, and HKU1), influenzavirus, dengue virus, RS virus, adenovirus, varicella-zoster virus, herpes simplex virus, measles virus, parainfluenza virus, enterovirus, rhinovirus, and human metapneumovirus.

(29) The adrenomedullin or a derivative thereof, or an antiviral agent for use according to the embodiment (27) or (28), wherein the viral infection is viral pneumonia, viral myocarditis, viral encephalitis, viral hemorrhagic fever, viral nephropathy, viral gastroenteritis, viral vasculitis, viral stomatitis, viral keratitis, or viral neuritis, and the sequela after cure of the viral infection is dyspnea or shortness of breath remaining after cure of viral pneumonia.

(30) Use of the adrenomedullin or a derivative thereof defined in any of the embodiments (1) to (20), or the medicament according to any of the embodiments (1) to (20) in the manufacture of a medicament for the prevention or treatment of a symptom or disorder due to a viral infection, and a sequela after cure of a viral infection.

(31) Use of the adrenomedullin or a derivative thereof defined in any of the embodiments (1) to (20), or the medicament according to any of the embodiments (1) to (20) for the prevention or treatment of a symptom or disorder due to a viral infection, and a sequela after cure of a viral infection.

(32) The use according to the embodiment (30) or (31), wherein the viral infection is caused by one or more viruses selected from the group consisting of novel coronavirus (SARS-CoV-2), severe acute respiratory syndrome (SARS) coronavirus, Middle East respiratory syndrome (MERS) coronavirus, conventional human coronavirus (229E, NL63, OC43, and HKU1), influenzavirus, dengue virus, RS virus, adenovirus, varicella-zoster virus, herpes simplex virus, measles virus, parainfluenza virus, enterovirus, rhinovirus, and human metapneumovirus.

(33) The use according to any of the embodiments (30) to (32), wherein the viral infection is viral pneumonia, viral myocarditis, viral encephalitis, viral hemorrhagic fever, viral nephropathy, viral gastroenteritis, viral vasculitis, viral stomatitis, viral keratitis, or viral neuritis, and the sequela after cure of the viral infection is dyspnea or shortness of breath remaining after cure of viral pneumonia.

### Advantageous Effects of Invention

The invention can provide means to prevent and/or treat a symptom due to a viral infection in a subject affected by the viral infection, without causing any unwanted serious side effect, by optimizing a dosage and administration of a medicament comprising AM or a derivative thereof as an active ingredient.

The present specification includes contents described in the specification and/or drawings of Japanese patent application No. 2020-151099 to which the present application claims priority.

### BRIEF DESCRIPTION OF DRAWINGS

FIG. 1 shows graphs representing time course of blood pressure for cases 2 and 8 in a group with long-term mechanical ventilation for 9 to 15 days in Test IV, wherein the cases showed relatively stable blood pressure during continuous administration for 72 hours. In the figure, A and B show the results for case 2 and case 8, respectively. In the figure, BP, DBP, SBP, and PR indicate blood pressure, diastolic blood pressure, systolic blood pressure, and pulse rates, respectively. The abscissa represents elapsed time (hour), and the ordinate represents BP (mmHg) or PR (bpm).

FIG. 2 shows graphs representing time course of blood pressure for cases 3, 5, and 10 in a group with short duration of mechanical ventilation of around 5 days in Test IV, wherein the cases showed relatively stable blood pressure during continuous administration for 72 hours. In the figure, A, B, and C show the results for case 3, case 5, and case 10, respectively. In the figure, BP, DBP, SBP, and PR indicate blood pressure, diastolic blood pressure, systolic blood pressure, and pulse rates, respectively. The abscissa represents elapsed time (hour), and the ordinate represents BP (mmHg) or PR (bpm).

### DESCRIPTION OF EMBODIMENTS

Adrenomedullin (AM) is a bioactive peptide which was isolated and identified from pheochromocytoma in 1993 (Non Patent Literature 1). At the beginning of the discovery, AM was found to exert a strong vasodilatory hypotensive effect. Subsequent studies revealed that AM exerts diverse pharmacological effects such as a cardiovascular protective effect, an anti-inflammatory effect, an angiogenic effect, a tissue repair promoting effect, and an organ protective effect. In an effort to apply the pharmacological effects of AM to treatment of disease, administration of AM to patients with different diseases has been attempted.

AM is known to have a cardiovascular protective effect, an anti-inflammatory effect, an angiogenic effect, a tissue repair promoting effect, an organ protective effect, or the like. Hence, the preventive and/or therapeutic effects of AM against symptoms or disorders, such as organ dysfunctions, in patients with viral infections were considered promising. However, AM has a strong vasodilatory hypotensive effect. Thus, from the viewpoint of management of blood pressure, it has been needed for long-term administration of AM to avoid administration at night, in which reduction in blood pressure is expected. In administering AM to patients with viral infections, excessively high rates of administration or excessive doses may cause reduction in blood pressure due to the excessive vasodilatory effect of AM, leading to a shock state. For this reason, it has been needed in administering AM to patients with viral infections to optimize a dosage and administration.

The present inventors have found that by administering a specific dose of AM to a patient with viral pneumonia, one of viral infections, according to a specific administration regimen, symptoms or disorders due to viral pneumonia can be prevented and/or treated without causing any unwanted serious side effect. Therefore, an aspect of the invention relates to a medicament for preventing or treating a symptom or disorder in a subject affected by a viral infection, which comprises AM or a derivative thereof as an active ingredient.

In each aspect of the invention, the “derivative of adrenomedullin” (AM derivative) has adrenomedullin activity. In each aspect of the invention, the term “adrenomedullin or a derivative thereof” encompasses, not only “native adrenomedullin” described later, but also, for example, an adrenomedullin analog as a peptide consisting of an amino acid sequence of adrenomedullin with a part thereof deleted, substituted, or added; a peptide consisting of an amino acid sequence of adrenomedullin or an adrenomedullin analog and having a disulfide bond formed by two cysteine residues in the amino acid sequence, or a peptide wherein the disulfide bond of that peptide is substituted with an ethylene group; and a compound having, as its substructure, a peptide chain corresponding to AM or a peptide chain corresponding to an AM analog.

Herein, the term adrenomedullin activity means various physiological effects shown as examples in (1) to (10) below, in particular, means physiological effects that can be involved in prevention or treatment of symptoms or disorders of viral infections, such as a preventive or repairing effect for organ dysfunctions via an anti-inflammatory effect, an angiogenic effect, a tissue repair promoting effect, an organ protective effect, or the like. The term adrenomedullin activity also means an effect of increasing the cAMP amount in a subject by binding to an adrenomedullin receptor (CLR/RAMP2 or CLR/RAMP3) in the subject, and an effect of causing bioactivity in a subject by binding to an adrenomedullin receptor (CLR/RAMP2 or CLR/RAMP3) in the subject. The medicament according to the aspect, even when containing a derivative of AM as described above as an active ingredient, can prevent or treat symptoms or disorders due to viral infections in subjects affected by the viral infections, for example, in human patients affected by the viral infections, and sequelae thereof via bioactivity substantially approximately equivalent to that of native adrenomedullin (i.e., adrenomedullin activity).

(1) Cardiovascular: a vasodilatory effect, an effect of lowering blood pressure, an effect of suppressing increase in blood pressure, an effect of increasing cardiac output or improving cardiac insufficiency, an effect of improving pulmonary hypertension, an angiogenic effect, a lymphangiogenic effect, an effect of improving vascular endothelial function, control of vasopermeability, control of endothelial intercellular adhesion, an endothelial barrier protection effect, an antiarteriosclerotic effect, a myocardial protective effect (such as a myocardial protective effect in ischemic reperfusion disorder or inflammation), an effect of suppressing postmyocardial remodeling, an effect of suppressing cardiac hypertrophy, and an effect of suppressing an angiotensin-converting enzyme.

(2) Kidney and water and electrolyte system: a diuretic effect, a natriuretic effect, an effect of suppressing antidiuretic hormone, an aldosterone-reducing effect, a renoprotective effect (such as a myocardial protective effect in high blood pressure or ischemic reperfusion disorder), an effect of suppressing diabetic renal disease, an effect of suppressing C3 nephropathy, an effect of suppressing drinking behavior, and an effect of suppressing salt requirement.

(3) Brain and nervous system: an effect of neuroprotection and suppressing encephalopathy, an anti-inflammatory effect, an effect of suppressing apoptosis (such as an effect of suppressing apoptosis in ischemic reperfusion disorder or inflammation), an effect of maintaining autoregulatory capacity, an effect of suppressing oxidative stress, an effect of improving dementia, and a sympathoinhibitory effect.

(4) Urogenital: an effect of improving erection, an effect of improving blood flow, and an implantation-promoting effect.

(5) Gastrointestinal system: an antiulcer effect, a tissue repair effect, an effect of neogenesis of mucous membrane, an intestinal barrier protection effect, an effect of improving blood flow, an anti-inflammatory effect, and an effect of improving liver function.

(6) Orthopedics: an effect of stimulating osteoblast and an effect of improving arthritis.

(7) Endocrine metabolic system: an adipocyte-differentiating effect, an effect of regulating lipolysis, an effect of improving insulin sensitivity, an effect of controlling insulin secretion, an effect of suppressing antidiuretic hormone secretion, and an effect of suppressing aldosterone secretion.

(8) Respiratory system: a bronchodilating effect, a lung protection effect, an effect of improving emphysema, suppression of pulmonary fibrogenesis, suppression of pneumonia, an effect of suppressing bronchitis, and an effect of improving respiration.

(9) Immune system: an effect of promoting decomposition of C3b.

(10) Other: an effect of improving circulation, an anti-inflammatory effect, an effect of modulating cytokine, an organ protective effect, an effect of suppressing oxidative stress, a tissue repair effect (such as an anti-decubitus effect), an effect of improving septicemia, an effect of improving septic shock, an effect of suppressing multiple organ failure, an effect of suppressing fibrogenesis, an effect of suppressing auto-immune disease, an effect of suppressing diabetic retinopathy, an antimicrobial effect, a hair growth effect, and a pilatory effect.

In each aspect of the invention, the viral infection to be prevented or treated is caused by, for example, one or more viruses selected from the group consisting of novel coronavirus (SARS-CoV-2), severe acute respiratory syndrome (SARS) coronavirus, Middle East respiratory syndrome (hereinafter, also described as “MERS”) coronavirus, conventional human coronavirus (229E, NL63, OC43, and HKU1), influenzavirus, dengue virus, RS virus, adenovirus, varicella-zoster virus, herpes simplex virus, measles virus, parainfluenza virus, enterovirus, rhinovirus, and human metapneumovirus, in particular, caused by one or more viruses selected from the group consisting of SARS-CoV-2 and influenzavirus. For the viruses shown as examples, except a few viruses such as influenzavirus and herpesvirus, no effective therapeutic agent has been established yet. Viral infections caused by influenzavirus, varicella-zoster virus, and measles virus are each expected to be prevented by vaccination against the corresponding virus. However, even influenzavirus affects and kills many people every year in the world. Also in Japan, thousands of people die of viral infections caused by influenzavirus every year. AM or a derivative thereof as an active ingredient of the medicament or the like according to the aspect has a preventive or therapeutic effect against symptoms or disorders due to viral infections caused by those viruses. Accordingly, by administering the medicament or the like comprising AM or a derivative thereof as an active ingredient to a subject affected by a viral infection caused by any of the viruses shown as examples, for example, a human patient affected by any of the viruses shown as examples, the symptom or disorder due to the viral infection can be prevented or treated.

In each aspect of the invention, examples of the viral infection in a subject affected by the viral infection, for example, a human patient affected by the viral infection include, but are not limited to, viral pneumonia, viral myocarditis, viral encephalitis, viral hemorrhagic fever, viral nephropathy, viral gastroenteritis, viral vasculitis, viral stomatitis, viral keratitis, and viral neuritis, in particular, viral pneumonia. Viral pneumonia in a subject affected by viral pneumonia, for example, a human patient affected by viral pneumonia is typically viral severe pneumonia, in particular, viral severe pneumonia for which mechanical ventilation is needed. Herein, “severe pneumonia” means pneumonia such that a symptom of respiratory failure or the like is found. Herein, “mechanical ventilation” means mechanically ensuring respiration by using a ventilator. In each aspect of the invention, by administering the medicament or the like comprising AM or a derivative thereof as an active ingredient to a subject affected by any of the viral infections shown as examples, for example, a human patient affected by any of the viruses shown as examples, the symptom or disorder due to the viral infection can be prevented or treated.

Examples of the symptom or disorder due to a viral infection in a subject affected by the viral infection, for example, a human patient affected by the viral infection include, but are not limited to: for viral pneumonia, organ dysfunctions, respiratory disorder (such as cough or dyspnea), fever, muscle aches, general malaise, septicemia, septic shock, and multi-organ failure (such as organ failure of the lung, kidney, liver, heart, blood vessels, or cerebral nerves); for viral myocarditis, myocarditis; for viral encephalitis, encephalitis; for viral hemorrhagic fever, hemorrhagic fever; for viral nephropathy, nephropathy; for viral gastroenteritis, gastroenteritis; for viral vasculitis, vasculitis; for viral stomatitis, stomatitis; for viral keratitis, keratitis; and for viral neuritis, neuritis. In each aspect of the invention, by administering the medicament or the like comprising AM or a derivative thereof as an active ingredient to a subject affected by a viral infection, for example, a human patient affected by a viral infection, a preventive or therapeutic effect for any of the symptoms or disorders due to viral infections shown as examples can be obtained via the anti-inflammatory effect, angiogenic effect, tissue repair promoting effect, organ protective effect, or the like, of AM.

Examples of unwanted serious side effects that can be generated in administering AM or a derivative thereof to a subject affected by a viral infection, for example, a human patient affected by a viral infection include, but are not limited to, circulatory failure (shock) due to excessive reduction in blood pressure. In each aspect of the invention, by administering the medicament or the like comprising AM or a derivative thereof as an active ingredient to a subject affected by a viral infection, for example, a human patient affected by a viral infection, the symptom or disorder due to the viral infection can be prevented or treated without causing any of the unwanted serious side effects shown as examples.

In each aspect of the invention, the preventive or therapeutic effect for a symptom or disorder due to a viral infection in a subject affected by the viral infection, for example, a human patient affected by the viral infection can be determined, for example, in such a manner that the medicament or the like comprising AM or a derivative thereof as an active ingredient is administered to a subject affected by a viral infection and a symptom or disorder due to the viral infection in the subject is evaluated, for example, with change in duration of treatment (such as duration in which mechanical ventilation was needed), clinical condition, clinical examination (such as hematology test and blood biochemical test), vital signs (such as blood pressure and heart rates), and proinflammatory cytokines or the like (such as IL-113, IL-6, IL-8, IL-10, TNF-α, IFN-γ, autotaxin, FDP, D-dimer, tPA, PIC, and PTX3), as indicators. In each aspect of the invention, the onset of an unwanted serious side effect (such as circulatory failure (shock) due to excessive reduction in blood pressure) in administering the medicament or the like comprising AM or a derivative thereof as an active ingredient to a subject affected by a viral infection, for example, a human patient affected by a viral infection can be determined, for example, through observation of hemodynamics such as blood pressure.

In the present specification, “prevention” means that onset (development or occurrence) of a symptom or disorder in a subject affected by a viral infection, for example, a human patient affected by a viral infection will be substantially precluded. In the present specification, “treatment” means suppression (such as suppression of progression), remission, restoration, and/or cure of a symptom or disorder that has appeared (developed or occurred), or a sequela remaining after cure of a viral infection in a subject affected by the viral infection, for example, a human patient affected by the viral infection.

In each aspect of the invention, AM may be not only a human-derived peptide isolated and identified from human pheochromocytoma (SEQ ID NO: 1, Non Patent Literature 1), but also a peptide (ortholog) derived from another non-human mammal (such as a warm-blooded animal) such as a pig (SEQ ID NO: 4), dog (SEQ ID NO: 6), cattle (SEQ ID NO: 8), rat (SEQ ID NO: 10), or mouse (SEQ ID NO: 12). In a living body, each of these peptides has a disulfide bond formed by two cysteine residues in the amino acid sequence, and is amidated at the C-terminus thereof. Herein, a peptide that is any of those peptides and has a disulfide bond and a C-terminal amide group is also described as “native adrenomedullin”, or simply as “adrenomedullin”. In each aspect of the invention, any of those peptides can be applied as an active ingredient.

In the present specification, “C-terminal amidation” means an aspect of post-translational modification of a peptide in a living body, and specifically means a reaction in which the main chain carboxyl group of C-terminal amino acid residue of the peptide is converted into an amide group. In the present specification, “formation of a disulfide bond between cysteine residues” or “disulfide bond formation by cysteine residues” means an aspect of post-translational modification of a peptide in a living body, and specifically means a reaction in which two cysteine residues in an amino acid sequence of a peptide form a disulfide bond (—S—S—). Many bioactive peptides produced in a living body are initially biosynthesized as a precursor protein with larger molecular weight. The precursor protein is subject to post-translational modifications, such as C-terminal amidation and/or disulfide bond formation by cysteine residues, during a process of intracellular transport to give a mature bioactive peptide. The C-terminal amidation typically proceeds by a C-terminal amidating enzyme that acts on a precursor protein. For a bioactive peptide having a C-terminal amide group, the precursor protein has a Gly residue bound to a C-terminal carboxyl group to be amidated and the Gly residue is converted into a C-terminal amide group by the C-terminal amidating enzyme. The C-terminal propeptide in the precursor protein has a repeat sequence comprising a combination of basic amino acid residues, such as Lys-Arg or Arg-Arg (Mizuno, Journal of Japanese Biochemical Society, 61(12): 1435-1461 (1989)). Disulfide bond formation by cysteine residues can proceed under oxidative conditions. Disulfide bond formation by cysteine residues in a living body typically proceeds by a protein disulfide isomerase that acts on the precursor protein.

In each aspect of the invention, the adrenomedullin or a derivative thereof is preferably a peptide or compound or a pharmaceutically acceptable salt thereof, or a pharmaceutically acceptable solvate thereof, the peptide or compound being selected from the group consisting of:
(i) a peptide consisting of an amino acid sequence of adrenomedullin,
(ii) a peptide consisting of an amino acid sequence of adrenomedullin and having a disulfide bond formed by two cysteine residues in the amino acid sequence,
(iii) a peptide wherein the disulfide bond of the peptide of (ii) is substituted with an ethylene group,
(iv) a peptide wherein 1 to 15 amino acid residues of any of the peptides of (i) to (iii) are deleted, substituted, or added,
(v) a peptide wherein any of the peptides of (i) to (iv) is amidated at the C-terminus thereof, (vi) a peptide wherein any of the peptides of (i) to (iv) has a glycine residue added to the C-terminus thereof, and
(vii) a compound represented by formula (A-I):

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (i) to (vi),
provided that modifying group A is bound to the N-terminal amino group of peptide group B with or without intervening linking group L.

In formula (A-I), modifying group A can be a modifying group selected from the group consisting of an organic group comprising a C₄-C₃₀ alkyl group, a C₄-C₃₀ alkenyl group or a polyethylene glycol group, an Fc region of an immunoglobulin, and serum albumin.

In an embodiment, the adrenomedullin or a derivative thereof is more preferably a peptide or compound or a pharmaceutically acceptable salt thereof, or a pharmaceutically acceptable solvate thereof, the peptide or compound being selected from the group consisting of:
(i) a peptide consisting of an amino acid sequence of adrenomedullin,
(ii) a peptide consisting of an amino acid sequence of adrenomedullin and having a disulfide bond formed by two cysteine residues in the amino acid sequence,
(v) a peptide wherein the peptide of (i) or (ii) is amidated at the C-terminus thereof,
(vi) a peptide wherein the peptide of (i) or (ii) has a glycine residue added to the C-terminus thereof, and
(vii) a compound represented by formula (A-I):

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (i), (ii), (v), and (vi),
provided that modifying group A is bound to the N-terminal amino group of peptide group B with or without intervening linking group L.

In formula (A-I), modifying group A can be a modifying group selected from the group polyethylene glycol group, an Fc region of an immunoglobulin, and serum albumin.

Among the peptides of (i) to (vi), the peptide set forth in (v), consisting of an amino acid sequence of adrenomedullin, being amidated at the C-terminal thereof, and having a disulfide bond formed by two cysteine residues in the amino acid sequence, represents a mature native adrenomedullin. The peptide of (i), consisting of an amino acid sequence of adrenomedullin, represents a form of native adrenomedullin prior to post-translational modification including C-terminal amidation and disulfide bond formation by cysteine residues (i.e., an immature form).

The peptide of (ii) can be formed by oxidizing thiol groups of two cysteine residues in the peptide of (i) with air or with a suitable oxidizing agent to form a disulfide bond. The peptide of (ii) can be used to establish a conformation of the peptide similar to that of native adrenomedullin. This similar conformation can lead adrenomedullin activity of the peptide of (ii) to an activity substantially approximately equivalent to that of native adrenomedullin.

The peptide of (iii) can be formed by converting a disulfide bond in the peptide of (ii) into an ethylene group. The substitution of a disulfide bond to an ethylene group can be accomplished by any method well known in the art (O. Keller et al., Helv. Chim. Acta, 1974, Volume 57, p. 1253). The peptide of (iii) can be used to stabilize a conformation of the peptide. Thus, the peptide of (iii) can sustainably exert adrenomedullin activity in a living body.

In a certain embodiment, the number of amino acid residues deleted, substituted, or added in the peptide of (iv) ranges, for example, from 1 to 15, from 1 to 10, from 1 to 8, from 1 to 5, or from 1 to 3. In a certain mode, the peptide of (iv) is an N-terminus-deleted peptide wherein amino acid residues at positions 1 to 15, positions 1 to 14, positions 1 to 13, positions 1 to 12, positions 1 to 11, positions 1 to 10, positions 1 to 9, positions 1 to 8, positions 1 to 7, positions 1 to 6, positions 1 to 5, positions 1 to 4, or positions 1 to 3 from the N-terminal side are deleted in any peptide of (i) to (iii). In a certain embodiment, the peptide of (iv) is an N-terminus-deleted peptide wherein amino acid residues at positions 1 to 15, positions 1 to 12, positions 1 to 10, positions 1 to 5, or positions 1 to 3 from the N-terminal side are deleted in any peptide of (i) to (iii). The N-terminus-deleted peptide may have further deletion, substitution, or addition of one or more (such as 1 to 5, 1 to 3, or 1 or 2) amino acid residues. The peptide of (iv) can be used to lead adrenomedullin activity of the peptide to an activity substantially approximately equivalent to that of native adrenomedullin. In addition, the peptide of (iv) can be used to allow the peptide to sustainably exert adrenomedullin activity in a living body.

In a particular embodiment, the N-terminus-deleted peptide of (iv) is a peptide wherein amino acid residues at positions 1 to 12 from the N-terminal side are deleted in any peptide of (i) to (iii) (hereinafter, also referred to as “AM(13-52)”), and can be a peptide wherein one to three amino acid residues are additionally substituted or deleted.

(SEQ ID NO: 14)
AM(13-52) S-F-G-C-R-F-G-T-C-T-V-Q-K-L-A-H-Q-I-Y-Q-
F-T-D-K-D-K-D-N-V-A-P-R-S-K-I-S-P-Q-G-Y

When peptide (iv) is AM(13-52), the disulfide bond in peptide (ii) is formed between a cysteine residue at position 4 and a cysteine residue at position 9 in the amino acid sequence of SEQ ID NO: 14. The one to three amino acid residues to be additionally substituted or deleted are each neither the cysteine residue at position 4 nor the cysteine residue at position 9 in the amino acid sequence of SEQ ID NO: 14, and are any one to three of amino acid residues at positions 29 to 35. Peptide (iv) can be, for example, a peptide wherein an amino acid residue at position 32 or 33 in the amino acid sequence of SEQ ID NO: 14 is substituted. Peptide (iv) can be a peptide wherein an amino acid residue at position 32 in the amino acid sequence of SEQ ID NO: 14 is substituted.

Peptide (iv) can be a peptide consisting of an amino acid sequence of any of SEQ ID NOs: 15 to 22.

(SEQ ID NO: 15, “*R” denotes D-arginine)
S-F-G-C-R-F-G-T-C-T-V-Q-K-L-A-H-Q-I-Y-Q-F-T-D-K-D-
K-D-N-V-A-P-*R-S-K-I-S-P-Q-G-Y
(SEQ ID NO: 16)
S-F-G-C-R-F-G-T-C-T-V-Q-K-L-A-H-Q-I-Y-Q-F-T-D-K-D-
K-D-N-V-A-P-K-S-K-I-S-P-Q-G-Y
(SEQ ID NO: 17)
S-F-G-C-R-F-G-T-C-T-V-Q-K-L-A-H-Q-I-Y-Q-F-T-D-K-D-
K-D-N-V-A-P-A-S-K-I-S-P-Q-G-Y
(SEQ ID NO: 18)
S-F-G-C-R-F-G-T-C-T-V-Q-K-L-A-H-Q-I-Y-Q-F-T-D-K-D-
K-D-N-V-A-P-G-S-K-I-S-P-Q-G-Y
(SEQ ID NO: 19)
S-F-G-C-R-F-G-T-C-T-V-Q-K-L-A-H-Q-I-Y-Q-F-T-D-K-D-
K-D-N-V-A-P-S-K-I-S-P-Q-G-Y
(SEQ ID NO: 20)
S-F-G-C-R-F-G-T-C-T-V-Q-K-L-A-H-Q-I-Y-Q-F-T-D-K-D-
K-D-N-V-A-P-D-S-K-I-S-P-Q-G-Y
(SEQ ID NO: 21)
S-F-G-C-R-F-G-T-C-T-V-Q-K-L-A-H-Q-I-Y-Q-F-T-D-K-D-
K-D-N-V-A-P-F-S-K-I-S-P-Q-G-Y
(SEQ ID NO: 22)
S-F-G-C-R-F-G-T-C-T-V-Q-K-L-A-H-Q-I-Y-Q-F-T-D-K-D-
K-D-N-V-A-P-R-P-K-I-S-P-Q-G-Y

In the adrenomedullin or a derivative thereof according to the embodiment, the peptide is particularly preferably a peptide consisting of an amino acid sequence of any of SEQ ID NOs: 15 to 22, being amidated at the C-terminus thereof, and having a disulfide bond formed by a cysteine residue at position 4 and a cysteine residue at position 9, and especially preferably a peptide consisting of the amino acid sequence of SEQ ID NO: 17, being amidated at the C-terminus thereof, and having a disulfide bond formed by a cysteine residue at position 4 and a cysteine residue at position 9. The adrenomedullin or a derivative thereof according to the embodiment having the properties can exhibit significantly superior pharmacokinetics, for example, with respect to biological stability, to the parent compound AM while maintaining pharmacological effects substantially equivalent to those of AM.

The peptide of (vi) can be converted to the peptide of (v) by a C-terminal amidating enzyme which can convert a glycine residue at the C-terminus of the peptide of (vi) into a C-terminal amide group. Therefore, the peptide of (vi) can be administered to a subject to form the peptide amidated at the C-terminus thereof in a living body of the subject after a certain period of time. Thus, the peptide of (vi) can sustainably exert adrenomedullin activity in a living body.

In each aspect of the invention, “derivative of adrenomedullin” or “adrenomedullin derivative” can be a compound having, as its substructure, a peptide chain corresponding to AM. The compound of (vii), represented by formula (A-I), is an example of such compounds.

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (i) to (vi),
provided that modifying group A is bound to the N-terminal amino group of peptide group B with or without intervening linking group L.

Peptide group B can be a peptide group having a structure formed by eliminating one hydrogen atom from the N-terminal α-amino group of any of the peptides of (i) to (vi). The compound represented by formula (A-I) can sustainably exert adrenomedullin activity in a living body. The compound represented by formula (A-I) can be used as an active ingredient to prevent or treat a symptom or disorder due to a viral infection in a subject affected by a viral infection, for example, a human patient affected by a viral infection, and a sequela thereof through adrenomedullin activity of the compound while substantially avoiding the onset of unwanted side effects.

In formula (A-I), modifying group A can be a modifying group selected from the group polyethylene glycol (PEG) group, an Fc region of an immunoglobulin, and serum albumin. For example, modifying group A can be an modifying group selected from the group consisting of: an organic group comprising a C₄-C₃₀ alkyl group such as a palmitoyl group or a myristoyl group, a C₄-C₃₀ alkenyl group such as an oleyl group, or a PEG group; a sugar group such as a monovalent group derivatized from monosaccharide, disaccharide, oligosaccharide, or polysaccharide (such as a glycosyl group); and a peptide group such as a monovalent group derivatized from polyglycine, polyglutamic acid, polylysine, or polyaspartic acid (such as a monovalent group forming a bond via an N-terminal amino group, a C-terminal carboxyl group, or a side chain group).

When modifying group A is an organic group comprising a polyethylene glycol group, L can be a divalent linking group represented by formula (z):

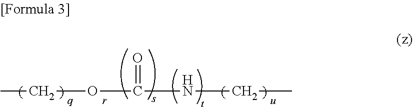

wherein
q and u are the same or different, and each represent an integer of 0 to 6, and
r, s, and t are the same or different, and each represent an integer of 0 or 1.

In this case, n is the integer 1.

The organic group comprising a polyethylene glycol (PEG) group comprises one or more PEG groups. The fashion of comprising one or more PEG groups is not limited. For example, one or more PEG groups may be disposed at a terminus of the organic group comprising a PEG group, or disposed in the inside of the organic group comprising a PEG group. The organic group comprising a PEG group has a weight-average molecular weight typically ranging from 1 to 2000 kDa, for example, ranging from 1 to 1000 kDa, preferably has a weight-average molecular weight ranging from 1 to 100 kDa, more preferably has a weight-average molecular weight ranging from 5 to 80 kDa, even more preferably has a weight-average molecular weight ranging from 10 to 80 kDa, or particularly preferably has a weight-average molecular weight ranging from 20 to 80 kDa or 30 to 60 kDa. In a certain mode, the organic group comprising a PEG group has a structure represented by any of formulas ((3) to (6):

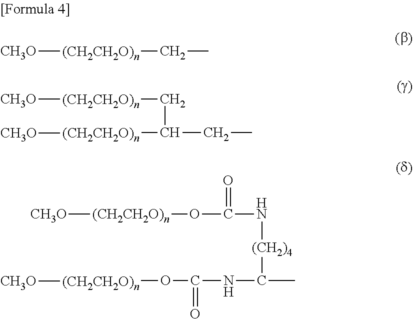

The organic group comprising a PEG group may be any of various groups known as a linear or branched group comprising a PEG group in the art in addition to the groups shown above. Examples of known groups that can be used as the organic group comprising a PEG group include, but are not limited to, groups disclosed in WO 1995/11924, WO 2006/084089, WO 98/41562, WO 2005/079838, WO 2002/060978, WO 2001/048052, WO 1998/055500, WO 1996/021469, WO 2003/040211, and JP H04-108827.

In a certain mode, in formula (z), u can be the integer 0. For the combination of q to t in this case, q can be an integer of 0 to 6, s and r can be each an integer of 0 or 1, t can be the integer 0, and u can be the integer 0. Alternatively, q can be an integer of 1 to 6, r, t, and u can be each the integer 0, and s can be the integer 1. A specific example of compounds in which the organic group comprising a polyethylene glycol group is bound to peptide group B via such a linking group is an amide-linked long-acting adrenomedullin derivative, for example, disclosed in the specification of WO 2015/141819.

In a certain embodiment, in formula (z), u can be an integer of 1 to 6. For the combination of q to u in this case, q and u can be the same or different, and each an integer of 1 to 6, and r, s, and t can be the same or different, and each an integer of 0 or 1. Alternatively, for the combination of q to u in formula (z), q can be the integer 0, u can be an integer of 1 to 6, and r, s, and t can be each the integer 1. A specific example of compounds in which the organic group comprising a polyethylene glycol group is bound to peptide group B via such a linking group is an alkylamine-linked long-acting adrenomedullin derivative, for example, disclosed in the specification of WO 2017/047788.

In a certain embodiment, in formula (z), q can be an integer of 0 to 6, r and s can be each the integer 1, and t and u can be each the integer 0. A specific example of compounds in which the organic group comprising a polyethylene glycol group is bound to peptide group B via such a linking group is an urethane-linked long-acting adrenomedullin derivative, for example, disclosed in the specification of WO 2017/047788.

When A is an Fc region of an immunoglobulin in formula (A-I), L can be a linking group consisting of a peptide having any amino acid sequence. In this case, n is the integer 1. A is preferably an Fc region of immunoglobulin G1 (IgG1), or an Fc region of immunoglobulin G4 (IgG4). An appropriate mammal from which the Fc region of an immunoglobulin for use as A in formula (A-I) is to be derived can be selected on the basis of a subject to which the medicament or the like according to the aspect is to be applied, which is described later. A is preferably an Fc region of an immunoglobulin derived from a human or a non-human mammal (e.g., a warm-blooded animal such as a pig, dog, cattle, rat, mouse, guinea pig, rabbit, chicken, sheep, cat, monkey, hamadryas baboon, or chimpanzee), and more preferably an Fc region of an immunoglobulin derived from a human or a non-human mammal of the same species as a subject to which the medicament or the like according to the aspect is to be applied.

Linking group L consisting of a peptide having any amino acid sequence is, for example, a linking group having an amino acid sequence that is, as n is defined as the number of repetitions, (GGGS)n (SEQ ID NO: 23) (n is an integer ranging from 2 to 10), (GGGS)n+GGGK (SEQ ID NOs: 23 and 24) (n is an integer ranging from 1 to 9, preferably an integer ranging from 2 to 6), or (GGGGS)n+GGGGK (SEQ ID NOs: 25 and 26) (n is an integer ranging from 1 to 6, preferably an integer ranging from 2 to 5). In a certain mode, linking group L consisting of a peptide can be a linking group consisting of a peptide having the following amino acid sequence:
GGGGSGGGGSGGGGS (SEQ ID NO: 27) (hereinafter, also referred to as “linking group Ls”); or
GGGGSGGGGSGGGGK (SEQ ID NO: 28) (hereinafter, also referred to as “linking group Lk”).

It is preferable that Fc region A and linking group L be linked to each other via a peptide bond formed by the C-terminal carboxyl group of the Fc region and the N-terminal α amino group of linking group L, and peptide group B and linking group L be linked to each other via a peptide bond formed by the N-terminal a amino group of the peptide group with the C-terminal carboxyl group of linking group L. That is, the compound represented by formula (A-I) according to the embodiment has a structure of protein or polypeptide, as a whole. A specific example of compounds wherein modifying group A is an Fc region and linking group L is a linking group consisting of a peptide is an immunoglobulin Fc region-linked long-acting adrenomedullin derivative, for example, disclosed in the specification of WO 2018/181638.

When modifying group L is an Fc region and linking group L is linking group Ls or linking group Lk, peptide group B can be a peptide group having a structure formed by eliminating one hydrogen atom from the N-terminal amino group of an N-terminus-deleted peptide wherein amino acid residues at positions 1 to 15, positions 1 to 14, positions 1 to 13, positions 1 to 12, positions 1 to 11, positions 1 to 10, positions 1 to 9, positions 1 to 8, positions 1 to 7, or positions 1 to 6 from the N-terminal side are deleted.

Specific examples of compounds represented by formula (A-I) wherein modifying group A is serum albumin include a serum albumin-linked long-acting adrenomedullin derivative, for example, disclosed in the specification of PCT/JP2021/29112 before being laid open.

The adrenomedullin or a derivative thereof is more preferably a peptide or compound or a pharmaceutically acceptable salt thereof, or a pharmaceutically acceptable solvate thereof, the peptide or compound being selected from the group consisting of:
(a) a peptide consisting of an amino acid sequence of SEQ ID NO: 1, or a peptide consisting of an amino acid sequence of SEQ ID NO: 1, and having a disulfide bond formed by a cysteine residue at position 16 and a cysteine residue at position 21;
(b) a peptide consisting of an amino acid sequence of SEQ ID NO: 4, or a peptide consisting of an amino acid sequence of SEQ ID NO: 4, and having a disulfide bond formed by a cysteine residue at position 16 and a cysteine residue at position 21;
(c) a peptide consisting of an amino acid sequence of SEQ ID NO: 6, or a peptide consisting of an amino acid sequence of SEQ ID NO: 6, and having a disulfide bond formed by a cysteine residue at position 16 and a cysteine residue at position 21;
(d) a peptide consisting of an amino acid sequence of SEQ ID NO: 8, or a peptide consisting of an amino acid sequence of SEQ ID NO: 8, and having a disulfide bond formed by a cysteine residue at position 16 and a cysteine residue at position 21;
(e) a peptide consisting of an amino acid sequence of SEQ ID NO: 10, or a peptide consisting of an amino acid sequence of SEQ ID NO: 10, and having a disulfide bond formed by a cysteine residue at position 14 and a cysteine residue at position 19;
(f) a peptide consisting of an amino acid sequence of SEQ ID NO: 12, or a peptide consisting of an amino acid sequence of SEQ ID NO: 12, and having a disulfide bond formed by a cysteine residue at position 14 and a cysteine residue at position 19;
(g) a peptide wherein the disulfide bond of any of the peptides of (a) to (f) is substituted with an ethylene group;
(h) a peptide wherein 1 to 15 amino acid residues of any of the peptides of (a) to (g) are deleted, substituted, or added;
(i) a peptide wherein any of the peptides of (a) to (h) is amidated at the C-terminus thereof;
(j) a peptide wherein any of the peptides of (a) to (h) has a glycine residue added to the C-terminus thereof; and
(k) a compound represented by formula (A-I):

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (a) to (j),
provided that modifying group A is bound to the N-terminal amino group of peptide group B with or without intervening linking group L.

In formula (A-I), modifying group A can be a modifying group selected from the group consisting of an organic group comprising a C₄-C₃₀ alkyl group, a C₄-C₃₀ alkenyl group or a polyethylene glycol group, an Fc region of an immunoglobulin, and serum albumin.

In an embodiment, the adrenomedullin or a derivative thereof is even more preferably a peptide or compound or a pharmaceutically acceptable salt thereof, or a pharmaceutically acceptable solvate thereof, the peptide or compound being selected from the group consisting of:
(a) a peptide consisting of an amino acid sequence of SEQ ID NO: 1, or a peptide consisting of an amino acid sequence of SEQ ID NO: 1, and having a disulfide bond formed by a cysteine residue at position 16 and a cysteine residue at position 21;
(b) a peptide consisting of an amino acid sequence of SEQ ID NO: 4, or a peptide consisting of an amino acid sequence of SEQ ID NO: 4, and having a disulfide bond formed by a cysteine residue at position 16 and a cysteine residue at position 21;
(c) a peptide consisting of an amino acid sequence of SEQ ID NO: 6, or a peptide consisting of an amino acid sequence of SEQ ID NO: 6, and having a disulfide bond formed by a cysteine residue at position 16 and a cysteine residue at position 21;
(d) a peptide consisting of an amino acid sequence of SEQ ID NO: 8, or a peptide consisting of an amino acid sequence of SEQ ID NO: 8, and having a disulfide bond formed by a cysteine residue at position 16 and a cysteine residue at position 21;
(e) a peptide consisting of an amino acid sequence of SEQ ID NO: 10, or a peptide consisting of an amino acid sequence of SEQ ID NO: 10, and having a disulfide bond formed by a cysteine residue at position 14 and a cysteine residue at position 19;
(f) a peptide consisting of an amino acid sequence of SEQ ID NO: 12, or a peptide consisting of an amino acid sequence of SEQ ID NO: 12, and having a disulfide bond formed by a cysteine residue at position 14 and a cysteine residue at position 19;
(i) a peptide wherein any of the peptide of (a) to (f) is amidated at the C-terminus thereof; (j) a peptide wherein any of the peptides of (a) to (f) has a glycine residue added to the C-terminus thereof; and
(k) formula (A-I):

A-Lₙ-B (A-I)

wherein
A is a modifying group,
L is a divalent linking group,
n is an integer of 0 or 1, and
B is a peptide group having a structure formed by eliminating one hydrogen atom from the N-terminal amino group of any of the peptides of (a) to (f), (i), and (j),
provided that modifying group A is bound to the N-terminal amino group of peptide group B with or without intervening linking group L.

In formula (A-I), modifying group A can be a modifying group selected from the group polyethylene glycol group, an Fc region of an immunoglobulin, and serum albumin.

The number of amino acid residues deleted, substituted, or added in the peptide of (h) preferably ranges from 1 to 12, more preferably ranges from 1 to 10, even more preferably ranges from 1 to 8, particularly preferably ranges from 1 to 5, and most preferably ranges from 1 to 3. A suitable peptide of (h) is a peptide wherein amino acid residues of any of the peptides of (a) to (g) at positions 1 to 15, positions 1 to 12, positions 1 to 10, positions 1 to 8, positions 1 to 5, or positions 1 to 3 from the N-terminal side are deleted, and a more suitable peptide of (h) is a peptide wherein amino acid residues of any of the peptides of (a) to (d) at positions 1 to 15, positions 1 to 10, or positions 1 to 5 from the N-terminal side are deleted, or a peptide wherein amino acid residues of the peptide of (e) or (f) at positions 1 to 13, positions 1 to 8, or positions 1 to 5 from the N-terminal side are deleted. The suitable peptide may have further deletion, substitution, or addition of one or more (such as 1 to 5, 1 to 3, or 1 or 2) amino acid residues. Th peptide of (h) can be used to lead adrenomedullin activity of the peptide to an activity substantially approximately equivalent to that of native adrenomedullin. In addition, the peptide of (h) can be used to allow the peptide to sustainably exert adrenomedullin activity in a living body.

In each aspect of the invention, the AM or a derivative thereof to be used as an active ingredient includes not only the compound itself but also a salt thereof. When the AM or a derivative thereof is in the form of salt, it is preferably a pharmaceutically acceptable salt. Counterions in a salt of the AM or a derivative thereof preferably include, but are not limited to, for example, cations such as a sodium, potassium, calcium, magnesium, or substituted or unsubstituted ammonium ion, or anions such as a chloride, bromide, iodide, phosphate, nitrate, sulfate, carbonate, bicarbonate, perchlorate, formate, acetate, trifluoroacetate, propionate, lactate, maleate, hydroxymaleate, methylmaleate, fumarate, adipate, benzoate, 2-acetoxybenzoate, p-aminobenzoate, nicotinate, cinnamate, ascorbate, pamoate, succinate, salicylate, bismethylenesalicylate, oxalate, tartrate, malate, citrate, gluconate, aspartate, stearate, palmitate, itaconate, glycolate, glutamate, benzenesulfonate, cyclohexylsulfamate, methanesulfonate, ethanesulfonate, isethionate, benzenesulfonate, p-toluenesulfonate, or naphthalenesulfonate ion. When the AM or a derivative thereof is in the form of salt with any of the counterions, adrenomedullin activity of the compounds can be substantially approximately equivalent to that of native adrenomedullin.

In each aspect of the invention, the AM or a derivative thereof to be used as an active ingredient includes not only the compound itself but also a solvate of the compound or a salt thereof. When the AM or a derivative thereof or a salt thereof is in the form of a solvate, it is preferably a pharmaceutically acceptable solvate. Solvents that can form solvates with the compound or a salt thereof preferably include, but are not limited to, for example, water or organic solvents such as methanol, ethanol, 2-propanol (isopropyl alcohol), dimethyl sulfoxide (DMSO), acetic acid, ethanolamine, acetonitrile, or ethyl acetate. When the AM or a derivative thereof or a salt thereof is in the form of solvate with any of the solvents described above, adrenomedullin activity of the compounds can be substantially approximately equivalent to that of native adrenomedullin.

In each aspect of the invention, the AM or a derivative thereof to be used as an active ingredient includes individual enantiomer and diastereomer of the compounds, and mixtures of stereoisomeric forms of the compounds such as racemates.

The AM or a derivative thereof having the properties can prevent or treat symptoms or disorders due to viral infections in subjects affected by viral infections, for example, in human patients affected by viral infections while maintaining the pharmacological effects of native adrenomedullin.

For the medicament according to the aspect, the AM or a derivative thereof to be used as an active ingredient may be used alone or in combination with one or more pharmaceutically acceptable components. The medicament according to the aspect can be formulated into various dosage forms commonly used in the art depending on the desired mode of administration. Thus, the medicament according to the aspect can also be provided in the form of a pharmaceutical composition comprising adrenomedullin or a derivative thereof, and one or more pharmaceutically acceptable carriers. In the case of the embodiment, pharmaceutical compositions may comprise, in addition to the components described above, one or more pharmaceutically acceptable media (e.g., solvent such as sterile water or solution such as physiological saline) and additive agents including excipients, binders, vehicles, dissolution aids, preservatives, stabilizers, disintegrators, disintegration inhibitors, bulking agents, lubricants, surfactants, emulsifying agents, oily liquids (such as plant oil), suspending agents, buffering agents, soothing agents, antioxidants, sweetening agents, flavoring agents, and so forth.

The dosage form of the medicament according to the aspect is not particularly limited and may be a formulation for parenteral administration, a formulation for transmucosal (such as transnasal, sublingual, or trans-oral-mucosal), transdermal, transanal (intestinal infusion), or transvaginal administration, or a formulation for oral administration. The dosage form of the medicament according to the aspect may also be a formulation in unit dosage form or in multiple dosage form. Formulations for use in parenteral administration include, for example, injections such as sterile solutions or suspensions in water or any other pharmaceutically acceptable liquid. Additive agents that can be admixed into the injections include, but are not limited to, for example, vehicles such as physiological saline and isotonic solutions comprising glucose or other pharmaceutic aids (such as D-sorbitol, D-mannitol, or sodium chloride); dissolution aids such as alcohols (such as ethanol or benzyl alcohol), esters (such as benzyl benzoate), and polyalcohols (such as propylene glycol or polyethylene glycol); nonionic surfactants such as polysorbate 80 or polyoxyethylene hydrogenated castor oil; oily liquids such as sesame oil or soybean oil; buffering agents such as phosphate buffer or sodium acetate buffer; soothing agents such as benzalkonium chloride or procaine hydrochloride; stabilizers such as human serum albumin or polyethylene glycol; preservatives; and antioxidants. The prepared injection will be generally filled in any suitable container (such as a vial or an ampule) and preserved under an appropriate environment until use.

The medicament according to the aspect can be combined with one or more other drugs useful as medicaments. In this case, the medicament according to the aspect may be provided in the form of a single medicament comprising AM or a derivative thereof, and one or more other drugs, or may be provided in the form of a medicament combination or kit comprising a plurality of formulations into which AM or a derivative thereof, and one or more other drugs are separately formulated. For the medicament combination or kit, each formulation can be administered simultaneously or separately (such as sequentially).

For applying the AM or a derivative thereof to pharmaceutical use, the AM or a derivative thereof includes not only the compound itself but also a pharmaceutically acceptable salt thereof and a pharmaceutically acceptable solvate thereof. Examples of the pharmaceutically acceptable salts of AM or a derivative thereof and pharmaceutically acceptable solvates thereof preferably include, but are not limited to, salts or solvates exemplified above. When AM or a derivative thereof is in the form of any of the salts or solvates described above, the compound can be applied to the desired pharmaceutical use.

For applying the AM or a derivative thereof to pharmaceutical use, the AM or a derivative thereof includes not only the compound itself but also prodrug forms of the compound. In the present specification, “prodrug” means a compound that is converted into a parent drug in a living body. Examples of prodrug forms of the AM or a derivative thereof include, but are not limited to, esters of a hydroxyl group of the AM or a derivative thereof and any carboxylic acid, amides of the hydroxyl group and any amine, and amides of an amino group of the AM or a derivative thereof and any carboxylic acid. When the AM or a derivative thereof is in any of the prodrug forms, the pharmacokinetics in administering the prodrug form to a subject can be improved without substantially lowering the adrenomedullin activity of the parent drug.

The medicament according to the aspect can be used for preventing or treating a symptom or disorder due to a viral infection, or a sequela after cure of a viral infection in a subject affected by the viral infection, or a subject affected by the sequela after cure of the viral infection, for example, a human patient affected by the viral infection, or a human patient affected by the sequela after cure of the viral infection. For the medicament according to the aspect, the viral infection is, for example, viral pneumonia, viral myocarditis, viral encephalitis, viral hemorrhagic fever, viral nephropathy, viral gastroenteritis, viral vasculitis, viral stomatitis, viral keratitis, or viral neuritis. In an embodiment, when the medicament according to the aspect is applied to a subject affected by viral pneumonia, for example, a human patient affected by viral pneumonia, the medicament according to the aspect is preferably used for preventing or treating organ dysfunctions, respiratory disorder (such as cough or dyspnea), fever, muscle aches, general malaise, septicemia, septic shock, or multi-organ failure (such as organ failure of the lung, kidney, liver, heart, blood vessels, or cerebral nerves) in the subject or human patient, and more preferably used for preventing or treating organ dysfunctions in the subject or human patient. Use for a sequela (dyspnea or shortness of breath) remaining after cure of viral pneumonia is preferred. In another embodiment, when the medicament according to the aspect is applied to a subject affected by viral myocarditis, for example a human patient affected by viral myocarditis, the medicament according to the aspect is preferably used for preventing or treating myocarditis in the subject or human patient. In another embodiment, when the medicament according to the aspect is applied to a subject affected by viral encephalitis, for example, a human patient affected by viral encephalitis, the medicament according to the aspect is preferably used for preventing or treating encephalitis in the subject or human patient. In another embodiment, when the medicament according to the aspect is applied to a subject affected by viral hemorrhagic fever, for example, a human patient affected by viral hemorrhagic fever, the medicament according to the aspect is preferably used for preventing or treating hemorrhagic fever in the subject or human patient. In another embodiment, when the medicament according to the aspect is applied to a subject affected by viral nephropathy, for example, a human patient affected by viral nephropathy, the medicament according to the aspect is preferably used for preventing or treating nephropathy in the subject or human patient. In another embodiment, when the medicament according to the aspect is applied to a subject affected by viral gastroenteritis, for example, a human patient affected by viral gastroenteritis, the medicament according to the aspect is preferably used for preventing or treating gastroenteritis in the subject or human patient. In another embodiment, when the medicament according to the aspect is applied to a subject affected by viral vasculitis, for example, a human patient affected by viral vasculitis, the medicament according to the aspect is preferably used for preventing or treating vasculitis in the subject or human patient. In another embodiment, when the medicament according to the aspect is applied to a subject affected by viral stomatitis, for example, a human patient affected by viral stomatitis, the medicament according to the aspect is preferably used for preventing or treating stomatitis in the subject or human patient. In another embodiment, when the medicament according to the aspect is applied to a subject affected by viral keratitis, for example, a human patient affected by viral keratitis, the medicament according to the aspect is preferably used for preventing or treating keratitis in the subject or human patient. In another embodiment, when the medicament according to the aspect is applied to a subject affected by viral neuritis, for example, a human patient affected by viral neuritis, the medicament according to the aspect is preferably used for preventing or treating neuritis in the subject or human patient. By using the medicament according to the aspect for preventing or treating any of the symptoms or disorders due to a viral infection shown as examples, or a sequela after cure of a viral infection shown as examples in a subject affected by the viral infection, or a subject affected by the sequela after cure of the viral infection, for example, a human patient affected by the viral infection, or a human patient affected by the sequela after cure of the viral infection, the symptom or disorder due to the viral infection or the sequela after cure of the viral infection can be prevented or treated.

The AM or a derivative thereof to be used as an active ingredient of the medicament according to the aspect is derived from adrenomedullin, a natural bioactive peptide. Clinical trials are underway for medicaments comprising the AM or a derivative thereof as an active ingredient as therapeutic agents against various diseases and the like. Accordingly, the AM or a derivative thereof has been confirmed to be safe and of low toxicity. Therefore, the medicament according to the aspect can be applied to various subjects in need of prevention or treatment of a symptom or disorder due to a viral infection. The subjects are preferably human or non-human mammalian (such as warm-blooded animal including pig, dog, cattle, rat, mouse, guinea pig, rabbit, chicken, sheep, cat, monkey, hamadryas baboon, or chimpanzee) subjects or patients, and more preferably human patients. The medicament according to the aspect can be administered to the subjects to prevent or treat a symptom or disorder due to a viral infection in the subjects.

When the medicament according to the aspect is administered to a subject, particularly a human patient, the precise dosage and administration will be determined considering many factors including age and sex of the subject, the precise condition (such as severity) of the condition, disease, and/or disorder to be prevented or treated, and the route of administration. The therapeutically effective dosage and administration should be ultimately determined by the attending physician. Therefore, the AM or a derivative thereof, which is an active ingredient in the medicament according to the aspect, will be administered to the subject in a therapeutically effective dosage and administration (such as dose and route of administration). For example, when the medicament according to the aspect is administered to a subject, in particular, a human patient, a dose of the AM or a derivative thereof, or a pharmaceutically acceptable salt, or a pharmaceutically acceptable solvate thereof, which is used as an active ingredient, will usually range from 0.01 to 1000 μg/kg body weight/day, for example, range from 0.5 to 200 μg/kg body weight/day.

The medicament according to the aspect may be administered through any route of administration. For example, the medicament according to the aspect is preferably administered through a route such as intravenous administration, intestinal infusion, subcutaneous administration, intramuscular administration, oral administration, or intraperitoneal administration, more preferably intravenously administered, and even more preferably intravenously administered in a continuous manner.

It has been revealed that when the medicament according to the aspect is administered to a subject affected by a viral infection, for example, a human patient affected by a viral infection, symptoms or disorders due to the viral infection can be prevented or treated without causing any unwanted serious side effect (such as circulatory failure (shock) due to excessive reduction in blood pressure) by intravenously administering a specific dose of AM in a continuous manner according to a specific administration regimen.

The AM or a derivative thereof, which is an active ingredient of the medicament according to the aspect, is preferably intravenously administered in a continuous manner. In the case of the embodiment, the dosage form of the medicament according to the aspect is, for example, an injection such as a sterile solution or suspension in water or any other pharmaceutically acceptable liquid (such as physiological saline). The intravenous administration is preferably performed in a continuous manner at a rate ranging from 1.0 to 100.0 ng active ingredient/kg body weight/min in terms of AM, more preferably performed in a continuous manner at a rate ranging from 1.0 to 50.0 ng active ingredient/kg body weight/min, even more preferably performed in a continuous manner at a rate ranging from 1.0 to 20.0 ng active ingredient/kg body weight/min, and particularly preferably performed in a continuous manner at a rate ranging from 6.0 to 18.0 ng active ingredient/kg body weight/min. If the rate of administration of the AM or a derivative thereof exceeds the upper limit value, an unwanted serious side effect such as circulatory failure (shock) due to excessive reduction in blood pressure may be caused. If the rate of administration of the AM or a derivative thereof is less than the lower limit value, no sufficient therapeutic effect may be obtained. Therefore, symptoms or disorders due to viral infections can be prevented or treated, without causing any unwanted serious side effect, by intravenously administering the medicament according to the aspect in a continuous manner at a rate of administration in the range.

In the case of the embodiment, the intravenous administration is preferably performed with continuous administration for 72 hours followed by intermittent administration for 8 hours per day. The intravenous administration is preferably performed for 3 to 10 days after initiation of administration. If the period of administration of the AM or a derivative thereof is less than the lower limit value, no sufficient therapeutic effect may be obtained. Therefore, symptoms or disorders due to viral infections can be prevented or treated, without causing any unwanted serious side effect, by intravenously administering the medicament according to the aspect in a continuous manner for a period of administration in the range.

The medicament according to the aspect is intravenously administered in a continuous manner preferably at a rate ranging from 1.0 to 20.0 ng active ingredient/kg body weight/min, more preferably at a rate ranging from 6.0 to 18.0 ng active ingredient/kg body weight/min in terms of the AM or a derivative thereof as an active ingredient, and the intravenous administration is preferably performed on the basis of an administration regimen such that the intravenous administration is performed with continuous administration for 72 hours followed by intermittent administration for 8 hours per day for 3 to 10 days after initiation of administration. Symptoms or disorders due to viral infections can be prevented or treated, without causing any unwanted serious side effect, by intravenously administering the medicament according to the aspect in a continuous manner on the basis of the administration regimen.

The AM or a derivative thereof to be used as an active ingredient of the medicament according to the aspect can be used for preventing or treating a symptom or disorder due to a viral infection, or a sequela after cure of a viral infection in a subject affected by the viral infection, or a subject affected by the sequela after cure of the viral infection, for example, a human patient affected by the viral infection, or a human patient affected by the sequela after cure of the viral infection. Therefore, another aspect of the invention relates to an agent for preventing or treating a viral infection, the agent comprising AM or a derivative thereof as an active ingredient. The agent for prevention or treatment according to the aspect has the same properties as the above-described medicament according to the aspect. The agent for prevention or treatment according to the aspect can be used with the same dosage and administration as for the above-described medicament according to the aspect. The agent for prevention or treatment according to the aspect can be used for preventing or treating a symptom or disorder due to a viral infection in a subject affected by the viral infection, for example, a human patient affected by the viral infection. For the agent for prevention or treatment according to the aspect, the viral infection is, for example, viral pneumonia, viral myocarditis, viral encephalitis, viral hemorrhagic fever, viral nephropathy, viral gastroenteritis, viral vasculitis, viral stomatitis, viral keratitis, or viral neuritis. The symptom or disorder due to a viral infection for the agent for prevention or treatment according to the aspect is, for example: for viral pneumonia, organ dysfunctions, respiratory disorder (such as cough or dyspnea), fever, muscle aches, general malaise, septicemia, septic shock, or multi-organ failure (such as organ failure of the lung, kidney, liver, heart, blood vessels, or cerebral nerves), in particular, organ dysfunctions; for viral myocarditis, myocarditis; for viral encephalitis, encephalitis; for viral hemorrhagic fever, hemorrhagic fever; for viral nephropathy, nephropathy; for viral gastroenteritis, gastroenteritis; for viral vasculitis, vasculitis; for viral stomatitis, stomatitis; for viral keratitis, keratitis; and for viral neuritis, neuritis. The sequela after cure of a viral infection is, for example, dyspnea or shortness of breath remaining after cure of viral pneumonia. Any of the symptoms or disorders due to a viral infection shown as examples, or a sequela after cure of a viral infection shown as examples can be prevented or treated by administering an effective amount of the AM or a derivative thereof to a subject affected by the viral infection or a subject affected by the sequela after cure of the viral infection, for example, a human patient affected by the viral infection or a human patient affected by the sequela after cure of the viral infection.

Another aspect of the invention relates to a method for preventing or treating a symptom or disorder due to a viral infection, or a sequela after cure of a viral infection, the method comprising administering an effective amount of AM or a derivative thereof, or a pharmaceutically acceptable salt thereof, or a pharmaceutically acceptable hydrate thereof to a subject affected by the viral infection or a subject affected by the sequela after cure of the viral infection, for example, a human patient affected by the viral infection or a human patient affected by the sequela after cure of the viral infection. The AM or a derivative thereof, or a pharmaceutically acceptable salt thereof, or a pharmaceutically acceptable hydrate thereof to be administered in the method according to the aspect has the same properties as the active ingredient of the above-described medicament according to the aspect. The method according to the aspect can be performed with the same dosage and administration as for the above-described medicament according to the aspect. For the method according to the aspect, the viral infection is, for example, viral pneumonia, viral myocarditis, viral encephalitis, viral hemorrhagic fever, viral nephropathy, viral gastroenteritis, viral vasculitis, viral stomatitis, viral keratitis, or viral neuritis. The symptom or disorder due to a viral infection for the method according to the aspect is, for example: for viral pneumonia, organ dysfunctions, respiratory disorder (such as cough or dyspnea), fever, muscle aches, general malaise, septicemia, septic shock, or multi-organ failure (such as organ failure of the lung, kidney, liver, heart, blood vessels, or cerebral nerves), in particular, organ dysfunctions; for viral myocarditis, myocarditis; for viral encephalitis, encephalitis; for viral hemorrhagic fever, hemorrhagic fever; for viral nephropathy, nephropathy; for viral gastroenteritis, gastroenteritis; for viral vasculitis, vasculitis; for viral stomatitis, stomatitis; for viral keratitis, keratitis; and for viral neuritis, neuritis. The sequela after cure of a viral infection is, for example, dyspnea or shortness of breath remaining after cure of viral pneumonia. Any of the symptoms or disorders due to a viral infection shown as examples, or a sequela after cure of a viral infection shown as examples can be prevented or treated by administering an effective amount of the AM or a derivative thereof to a subject affected by the viral infection or a subject affected by the sequela after cure of the viral infection, for example, a human patient affected by the viral infection or a human patient affected by the sequela after cure of the viral infection.

Another aspect of the invention relates to AM or a derivative thereof, or a pharmaceutically acceptable salt thereof, or a pharmaceutically acceptable hydrate thereof for use in the prevention or treatment of a symptom or disorder due to a viral infection, or a sequela after cure of a viral infection in a subject affected by the viral infection or a subject affected by the sequela after cure of the viral infection, for example, a human patient affected by the viral infection or a human patient affected by the sequela after cure of the viral infection. Yet another aspect of the invention is use of AM or a derivative thereof, or a pharmaceutically acceptable salt thereof, or a pharmaceutically acceptable hydrate thereof in the manufacture of a medicament for the prevention or treatment of a symptom or disorder due to a viral infection, or a sequela after cure of a viral infection in a subject affected by the viral infection or a subject affected by the sequela after cure of the viral infection, for example, a human patient affected by the viral infection or a human patient affected by the sequela after cure of the viral infection. The compound or the like according to the aspect has the same properties as the active ingredient of the above-described medicament according to the aspect. The compound or the like according to the aspect can be used with the same dosage and administration as for the above-described medicament according to the aspect. For the compound or the like or use according to the aspect, the viral infection is, for example, viral pneumonia, viral myocarditis, viral encephalitis, viral hemorrhagic fever, viral nephropathy, viral gastroenteritis, viral vasculitis, viral stomatitis, viral keratitis, or viral neuritis. The symptom or disorder due to a viral infection for the compound or the like or use according to the aspect is, for example: for viral pneumonia, organ dysfunctions, respiratory disorder (such as cough or dyspnea), fever, muscle aches, general malaise, septicemia, septic shock, or multi-organ failure (such as organ failure of the lung, kidney, liver, heart, blood vessels, or cerebral nerves), in particular, organ dysfunctions; for viral myocarditis, myocarditis; for viral encephalitis, encephalitis; for viral hemorrhagic fever, hemorrhagic fever; for viral nephropathy, nephropathy; for viral gastroenteritis, gastroenteritis; for viral vasculitis, vasculitis; for viral stomatitis, stomatitis; for viral keratitis, keratitis; and for viral neuritis, neuritis. The sequela after cure of the viral infection is, for example, dyspnea or shortness of breath remaining after cure of viral pneumonia. Any of the symptoms or disorders due to a viral infection shown as examples, or a sequela after cure of a viral infection shown as examples can be prevented or treated by administering an effective amount of the AM or a derivative thereof to a subject affected by the viral infection or a subject affected by the sequela after cure of the viral infection, for example, a human patient affected by the viral infection or a human patient affected by the sequela after cure of the viral infection.

### EXAMPLES

Hereinafter, the present invention will be described further specifically with reference to Examples. However, the technical scope of the present invention is not intended to be limited by these Examples.

### <Test I: Organ Protective Effect of Adrenomedullin on Influenzavirus-Infected Mice (1)>

Drug efficacy test for influenzavirus-infected mice was conducted with use of AM to evaluate the effect of AM on organ dysfunctions in influenzavirus-infected mice.

### [I-1: Test Drug]

### (a) Active Ingredient

A freeze-dried powder of synthetic human adrenomedullin (a peptide consisting of the amino acid sequence of SEQ ID NO: 1, amidated at the C-terminus thereof, and having a disulfide bond formed by two cysteine residues in the amino acid sequence).

### (b) Medium

Aqueous solution of 3.75 w/v % mannitol-0.5 w/v % glycine.

### (c) Preparation of Specimens for Administration

To a vial containing the active ingredient, 1.4 mL of the medium was added to dissolve the active ingredient; thus, 1000 μM specimen solution was prepared. The specimen solution was aliquoted into 100-μL portions, which were cryopreserved at −20° C. The specimen solution was diluted with the medium to prepare 0.5 nmol/kg/hour (low concentration) or 5 nmol/kg/hour (high concentration) specimen solution. After preparation, 100 μL of each specimen solution was quickly loaded in an osmotic pump (ALZET Osmotic Pump: 1007D). The weight of the pump was measured before and after loading to confirm that the specimen solution was properly loaded. Each specimen solution was prepared at time of use.

### [1-2: Test Virus Strain]

### (a) Virus Strain and Host Cells Therefor

The influenzavirus PR8(A/PR/8/34 (H1N1)), a stored strain possessed by Department of Microbiology and Immunology, School of Medicine, Aichi Medical University, was used as a virus strain for use in the test.

### (b) Preparation of Solution of Virus to be Inoculated

The cryopreserved virus strain was thawed, and appropriately diluted with fresh DMEM culture medium. The diluted solution was added to a culture flask in which MDCK cells as host cells had been cultured. The host cells were cultured in a carbon dioxide incubator for 1 hour (37° C., CO₂ concentration: 5%) to allow the virus to be adsorbed on the host cells. Thereafter, the diluted solution of the virus was removed. Fresh DMEM culture medium containing 0.25% trypsin was added to the host cells, and the host cells were cultured in the carbon dioxide incubator until cytopathogenic effect (CPE) appeared. Thereafter, the supernatant containing the virus and the host cells were separately collected. The supernatant collected was centrifuged (2000 rpm, 780×g, 4° C., for 5 minutes), and the resulting supernatant was obtained as stock solution of the virus to be inoculated. The thus-obtained stock solution of the virus to be inoculated was aliquoted into 1-mL portions in serum tubes, and cryopreserved (−80° C.) until use.

Virus concentrations were measured with a specific procedure using the stock solution of the virus to be inoculated. The stock solution of the virus to be inoculated was diluted with fresh culture medium to a virus concentration of 2×10⁶ PFU/mL, giving solution of the virus to be inoculated.

### [1-3: Drug Efficacy Test on Influenzavirus-Infected Mice]

Twenty-seven 5-week-old female SPF mice (BALB/c type (BALB/c Cr Slc)) were purchased (Japan SLC, Inc.). After a preliminary growing period for 6 days, the mice were grouped into a control group, a group with administration of AM at low concentration, and a group with administration of AM at high concentration (eight mice in each group). To each mouse in each group under anesthesia with isoflurane, 1 mg/kg (5 mL/kg) butorphanol tartrate was subcutaneously administered. Thereafter, the back skin of the mouse was incised, and an osmotic pump was subcutaneously implanted. During a period between the day before virus inoculation and day 5 after inoculation, the specimen solution at low concentration (0.5 nmol/kg/hour) or high concentration (5 nmol/kg/hour) was subcutaneously administered at a flow rate of 0.5 μL/hour. To the mice in the control group without administration of AM, the medium was subcutaneously administered at the same flow rate. Each mouse in each group was grown in an animal room (controlled temperature: 18 to 28° C., controlled humidity: 30 to 80% RH, 12-hour light and 12-hour dark). On the day of virus inoculation, for the mice of the control group without administration of AM and the mice of the groups with administration of AM, 0.05 mL of the solution of the virus to be inoculated per individual (1×10⁵ PFU/mouse) was dropped into the nasal cavity by using a micropipette. Growing was continued until day 5 after inoculation, as the day after the inoculation was defined as day 1 after inoculation. The following items were evaluated over the growing period.

### (a) General Condition

General condition was observed once per day after the day of grouping on the basis of general condition scores shown in Table 1 in the following.

TABLE 1

| | Site of occurrence | | | |
|---|---|---|---|---|
| Score | (1) Eye | (2) Hair coat | (3) Behavior | (4) Others |
| 3 | Blepharosynechia | Very poor coat | Reduced locomotion | Respiratory failure, |
| | (condition such | | (condition such that | leanness, prone |
| | that eyelids do not | | no movement is | position, |
| | open) | | present even on | hypothermal |
| | | | contact) | condition |
| 2 | Loss of eyelid | Dull hair, | Reduced locomotion | Irregular |
| | reflex (condition | standing hair | (condition such that | respiration (clear), |
| | such that eyelids | | movement appears | leanness |
| | open on contact | | on contact) | |
| | but exhibit no | | | |
| | blink reflex) | | | |
| 1 | Closed eyelids | Slightly standing | Showing interests in | Irregular |
| | (eyelids open on | hair | surroundings, | respiration |
| | contact) | | responding to stimuli | (tachypnea or |
| | | | | bradypnea) |
| 0 | Normal (shiny | Normal (a good | Normal | Normal (without |
| | eyes) | coat) | | disorder of |
| | | | | respiration and |
| | | | | others) |

### (b) Body Weight

The body weights were measured every day from the day of inoculation.

### (c) Gross Lesion Examination of Lung

On day 5 after virus inoculation, anesthesia was performed with isoflurane, and the osmotic pump was then removed. Approximately 0.3 mL of blood was collected from the caudal vena cava by using a syringe (Terumo Corporation) treated with EDTA-2Na. The blood obtained was centrifuged (approximately 4° C., 3000 rpm, 2150×g, for 10 minutes) to collect plasma. Aprotinin was mixed in the plasma at a ratio of 20 μL of aprotinin to 100 μL of the plasma, and the resultant was cryopreserved (−80° C.). The animals subjected to blood collection were euthanized by bleeding from the abdominal aorta. Gross examination of lungs was performed separately for the left lung and the right lung on the basis of an evaluation method shown in Table 2 below. Thereafter, the left lung, the right lung, the kidneys, the liver, and the heart were excised, and weighted with an electronic balance. The heart excised was horizontally sectioned into three parts. The central part of the heart was soaked in 10% neutral buffered formalin and stored. The residual upper and lower parts of the heart were put in separate tubes, and cryopreserved (−80° C.). Each of the kidneys excised was horizontally sectioned into three parts. The right kidney parts were cryopreserved (−80° C.). The left kidney parts were soaked in 10% neutral buffered formalin and stored. The liver excised was vertically sectioned into two parts, each of which was further horizontally sectioned into three parts. One part of the liver was cryopreserved (−80° C.). The rest parts of the liver were soaked in 10% neutral buffered formalin and stored. For the lungs excised, a part of the left lung (approximately 10 to 20 mg) was cryopreserved (−80° C.). The residual part of the left lung was soaked in 10% neutral buffered formalin and stored. The right lung was used for virological examination.

TABLE 2

| Score | Finding in gross observation for lung lesion |
|---|---|
| 4 | Consolidation was found totally |
| 3 | Consolidation was found in ½ to ⅔ |
| 2 | Consolidation was found in ⅓ to ½ |
| 1 | Consolidation was found in from very few to ⅓ |
| 0 | No consolidation was found |

### (d) Histopathological Examination

The lung (left lung) excised was fixed, and a paraffin section was then produced for every case in each group in accordance with a conventional method. The paraffin sections were HE-stained. Microscopic examination was performed. Histopathological findings were scored in five grades. 0: no change (−), 1: very mild (±), 2: mild (+), 3: moderate (++), 4: severe (+++).

### (e) Statistical Processing

For body weights, general condition scores, gross findings for the lung, and histopathological examination, mean and standard error were calculated for each group. Significance test was performed between the control group and the group with administration of AM at low concentration or high concentration with use of Wilcoxon's rank sum test for general condition scores, gross findings for the lung, and histopathological examination. The significance level was set to 5%, and cases of less than 5% and those of less than 1% were separately shown.

### [1-4: Results]

Table 3 shows general condition scores for mice of different groups. Each value in the table is the mean and standard error of scores for the mice of the corresponding group.

TABLE 3
General condition score

| | Group | | |
|---|---|---|---|
| | | Group with administration | |
| | Control group | of adrenomedullin | |
| | Dose (nmol/kg/hour) | | |
| | 0 | 0.5 | 5 |
| Number of animals | 8 | 8 | 8 |
| Days after | | | |
| inoculation | | | |
| 0 | 0.0 ± 0.0 | 0.0 ± 0.0 | 0.0 ± 0.0 |
| 1 | 0.0 ± 0.0 | 0.0 ± 0.0 | 0.0 ± 0.0 |
| 2 | 0.0 ± 0.0 | 0.0 ± 0.0 | 0.0 ± 0.0 |
| 3 | 0.0 ± 0.0 | 0.0 ± 0.0 | 0.0 ± 0.0 |
| 4 | 0.0 ± 0.0 | 0.0 ± 0.0 | 0.0 ± 0.0 |
| 5 | 0.2 ± 0.1 | 0.2 ± 0.0 | 0.2 ± 0.1 |
| 6 | 0.6 ± 0.1 | 0.5 ± 0.1 | 0.3 ± 0.1 |

Table 4 shows body weight for mice of different groups. Each value in the table is the mean and standard error of body weight for the mice of the corresponding group.

TABLE 4
Body weight

| | Group | | |
|---|---|---|---|
| | | Group with administration of | |
| | Control group | adrenomedullin | |
| | Dose (nmol/kg/hour) | | |
| | 0 | 0.5 | 5 |
| Number of animals | 8 | 8 | 8 |
| Days after | | | |
| inoculation | | | |
| 0 | 17.8 ± 0.2 | 18.2 ± 0.3 | 18.1 ± 0.2 |
| 1 | 18.2 ± 0.2 | 18.3 ± 0.2 | 18.1 ± 0.2 |
| 2 | 18.1 ± 0.2 | 18.2 ± 0.3 | 18.2 ± 0.2 |
| 3 | 16.6 ± 0.4 | 16.9 ± 0.3 | 17.0 ± 0.4 |
| 4 | 16.0 ± 0.5 | 16.0 ± 0.4 | 16.4 ± 0.5 |
| 5 | 15.1 ± 0.6 | 15.2 ± 0.5 | 15.7 ± 0.6 |

Table 5 shows scores of gross lesion examination of the lungs for different groups. Each value in the table is the mean and standard error of scores of gross lesion examination of the lungs for the mice of the corresponding group. “*” in the table indicates the presence of significant difference from the control group (*: p<0.05).

TABLE 5
Score of gross lesion examination of lung

| | Group | | |
|---|---|---|---|
| | | Group with administration of | |
| | Control group | adrenomedullin | |
| | Dose (nmol/kg/hour) | | |
| | 0 | 0.5 | 5 |
| Number of animals | 8 | 8 | 8 |
| Lung | | | |
| Left lung | 3.0 ± 0.4 | 2.0 ± 0.4 | 1.8 ± 0.4 |
| Right lung | 2.5 ± 0.3 | 2.0 ± 0.4 | 1.4 ± 0.3* |

As demonstrated in Table 5, the gross lesion formation in the lung was significantly suppressed in the mice of the groups with administration of AM.

### <Test II: Organ Protective Effect of Adrenomedullin on Influenzavirus-Infected Mice (2)>

Drug efficacy test for influenzavirus-infected mice was conducted with use of AM and an AM analog to evaluate the effects of AM and the AM analog on organ dysfunctions in influenzavirus-infected mice.

### [II-1: Test Drug]

### (a) Active Ingredient

1: A freeze-dried powder of synthetic human adrenomedullin (hAM(1-52)) (a peptide consisting of the amino acid sequence of SEQ ID NO: 1, amidated at the C-terminus thereof, and having a disulfide bond formed by two cysteine residues in the amino acid sequence).

2: A freeze-dried powder of an adrenomedullin analog ([Ala-44]hAM(13-52)) (a peptide consisting of the amino acid sequence of SEQ ID NO: 17, amidated at the C-terminus thereof, and having a disulfide bond formed by two cysteine residues in the amino acid sequence).

S-F-G-C-R-F-G-T-C-T-V-Q-K-L-A-H-Q-I-Y-Q-F-T-D-K-D-
K-D-N-V-A-P-A-S-K-I-S-P-Q-G-Y-CONH₂

### (b) Medium

Aqueous solution of 3.75 w/v % mannitol-0.5 w/v % glycine.

### (c) Preparation of Specimens for Administration

To a vial containing the active ingredient, 0.95 mL of the medium was added to dissolve the active ingredient. The specimen solution was aliquoted into 40-4, portions, which were cryopreserved at −20 to 30° C. The specimen solution was thawed in use, and diluted with the medium to prepare 2 or 2 nmol/mL specimen solution.

### [II-2: Test Virus Strain]

The same influenzavirus strain and host cells therefor as used in Test I were used as a virus strain and host cells therefor for use in the test. The host cells and solution of the virus to be inoculated were prepared with the same procedure as in Test I.

### [II-3: Drug Efficacy Test on Influenzavirus-Infected Mice]

Forty-five 5-week-old female SPF mice (BALB/c type (BALB/c Cr Slc)) were purchased (Japan SLC, Inc.). After a preliminary growing period for 5 days, the mice were grouped into a control group, a group with administration of hAM(1-52) at low concentration, a group with administration of hAM(1-52) at high concentration, a group with administration of [Ala-44]hAM(13-52) at low concentration, and a group with administration of [Ala-44]hAM(13-52) at high concentration (eight mice in each group). During a period between the day before virus inoculation and day 4 after inoculation, the specimen solution in a liquid volume of 5 mL/kg body weight was administered via the tail vein once per day. To the mice in the control group without administration of AM, the medium was administered under the same conditions. Each mouse in each group was grown in an animal room (controlled temperature: 18 to 28° C., controlled humidity: 30 to 80% RH, 12-hour light and 12-hour dark). On the day of virus inoculation, for the mice of the control group without administration of AM and the mice of the groups with administration of AM or the AM analog, 0.05 mL of the solution of the virus to be inoculated per individual (1×10⁵ PFU/mouse) was dropped into the nasal cavity by using a micropipette. Growing was continued until day 5 after inoculation, as the day after the inoculation was defined as day 1 after inoculation. The same items as in Test I were evaluated with the same procedures as in Test I over the growing period.

### [II-4: Results]

Table 6 shows general condition scores for mice of different groups. Each value in the table is the mean and standard error of scores for the mice of the corresponding group. “*” in the table indicates the presence of significant difference from the control group (**: p<0.01).

TABLE 6
General condition score

| | Group | | |
|---|---|---|---|
| | | Group with | |
| | Control group | administration of hAM(1-52) | |
| | Dose (nmol/kg) | | |
| | 0 | 10 | 100 |
| Number of animals | 8 | 8 | 8 |
| 1 day before | 0.0 ± 0.0 | 0.0 ± 0.0 | 0.0 ± 0.0 |
| inoculation | | | |
| Days after | | | |
| inoculation | | | |
| 0 | 0.0 ± 0.0 | 0.0 ± 0.0 | 0.0 ± 0.0 |
| 1 | 0.0 ± 0.0 | 0.0 ± 0.0 | 0.0 ± 0.0 |
| 2 | 0.3 ± 0.0 | 0.3 ± 0.0 | 0.2 ± 0.1 |
| 3 | 0.5 ± 0.0 | 0.5 ± 0.0 | 0.5 ± 0.0 |
| 4 | 0.8 ± 0.0 | 0.8 ± 0.0 | 0.8 ± 0.0 |
| 5 | 1.0 ± 0.0 | 1.0 ± 0.0 | 1.0 ± 0.0 |
| | Group | | |
| | | Group with administration of | |
| | Control group | [Ala-44]hAM(13-52) | |
| | Dose (nmol/kg) | | |
| | 0 | 10 | 100 |
| Number of animals | 8 | 8 | 8 |
| 1 day before | 0.0 ± 0.0 | 0.0 ± 0.0 | 0.0 ± 0.0 |
| inoculation | | | |
| Days after | | | |
| inoculation | | | |
| 0 | 0.0 ± 0.0 | 0.0 ± 0.0 | 0.0 ± 0.0 |
| 1 | 0.0 ± 0.0 | 0.0 ± 0.0 | 0.0 ± 0.0 |
| 2 | 0.3 ± 0.0 | 0.2 ± 0.0 | 0.1 ± 0.0** |
| 3 | 0.5 ± 0.0 | 0.5 ± 0.0 | 0.5 ± 0.0 |
| 4 | 0.8 ± 0.0 | 0.8 ± 0.0 | 0.8 ± 0.0 |
| 5 | 1.0 ± 0.0 | 1.0 ± 0.0 | 1.0 ± 0.0 |

Table 7 shows body weight for mice of different groups. Each value in the table is the mean and standard error of body weight for the mice of the corresponding group.

TABLE 7
Body weight

| | Group | | |
|---|---|---|---|
| | | Group with | |
| | Control group | administration of hAM(1-52) | |
| | Dose (nmol/kg) | | |
| | 0 | 10 | 100 |
| Number of animals | 8 | 8 | 8 |
| 1 day before | 15.8 ± 0.2 | 15.9 ± 0.2 | 15.9 ± 0.2 |
| inoculation | | | |
| Days after | | | |
| inoculation | | | |
| 0 | 15.8 ± 0.2 | 16.0 ± 0.3 | 16.0 ± 0.3 |
| 1 | 16.2 ± 0.3 | 16.4 ± 0.3 | 16.9 ± 0.3 |
| 2 | 15.2 ± 0.3 | 15.3 ± 0.3 | 16.0 ± 0.3 |
| 3 | 14.2 ± 0.2 | 14.4 ± 0.3 | 14.9 ± 0.3 |
| 4 | 13.3 ± 0.2 | 13.4 ± 0.2 | 14.0 ± 0.3 |
| 5 | 12.9 ± 0.2 | 13.1 ± 0.2 | 13.2 ± 0.2 |
| | Group | | |
| | | Group with administration of | |
| | Control group | [Ala-44]hAM(13-52) | |
| | Dose (nmol/kg) | | |
| | 0 | 10 | 100 |
| Number of animals | 8 | 8 | 8 |
| 1 day before | 15.8 ± 0.2 | 15.8 ± 0.2 | 15.8 ± 0.2 |
| inoculation | | | |
| Days after | | | |
| inoculation | | | |
| 0 | 15.8 ± 0.2 | 15.7 ± 0.3 | 15.8 ± 0.2 |
| 1 | 16.2 ± 0.3 | 16.1 ± 0.3 | 16.2 ± 0.3 |
| 2 | 15.2 ± 0.3 | 15.1 ± 0.3 | 15.5 ± 0.2 |
| 3 | 14.2 ± 0.2 | 14.0 ± 0.3 | 14.6 ± 0.3 |
| 4 | 13.3 ± 0.2 | 13.2 ± 0.2 | 13.6 ± 0.2 |
| 5 | 12.9 ± 0.2 | 12.8 ± 0.2 | 12.9 ± 0.1 |

Table 8 shows scores of gross lesion examination of the lungs for different groups. Each value in the table is the mean and standard error of scores of gross lesion examination of the lungs for the mice of the corresponding group. “*” in the table indicates the presence of significant difference from the control group (**: p<0.01).

TABLE 8
Score of gross lesion examination of lung

| | Group | | |
|---|---|---|---|
| | | Group with | |
| | Control group | administration of hAM(1-52) | |
| | Dose (nmol/kg) | | |
| | 0 | 10 | 100 |
| Number of animals | 8 | 8 | 8 |
| Lung | | | |
| Left lung | 3.9 ± 0.1 | 2.9 ± 0.1** | 3.3 ± 0.3 |
| Right lung | 4.0 ± 0.0 | 2.4 ± 0.2** | 3.1 ± 0.2** |
| | Group | | |
| | | Group with administration of | |
| | Control group | [Ala-44]hAM(13-52) | |
| | Dose (nmol/kg) | | |
| | 0 | 10 | 100 |
| Number of animals | 8 | 8 | 8 |
| Lung | | | |
| Left lung | 3.9 ± 0.1 | 2.8 ± 0.2** | 2.6 ± 0.2** |
| Right lung | 4.0 ± 0.0 | 2.9 ± 0.1** | 2.5 ± 0.2** |

Table 9 shows virus concentrations in the lung for different groups. Each value in the table is the mean and standard error of virus concentration (×104 PFU) in the right lung for the mice of the corresponding group. “*” in the table indicates the presence of significant difference from the control group (**: p<0.01).

TABLE 9
Virus concentration in lung

| | Group | | |
|---|---|---|---|
| | | Group with | |
| | Control group | administration of hAM(1-52) | |
| | Dose (nmol/kg) | | |
| | 0 | 10 | 100 |
| Number of animals | 8 | 8 | 8 |
| Virus concentration | 97.8 ± 3.4 | 52.0 ± 4.8** | 62.3 ± 8.7** |
| (×10⁴ PFU/right | | | |
| lung) | | | |
| | Group | | |
| | | Group with administration of | |
| | Control group | [Ala-44]hAM(13-52) | |
| | Dose (nmol/kg) | | |
| | 0 | 10 | 100 |
| Number of animals | 8 | 8 | 8 |
| Virus concentration | 97.8 ± 3.4 | 53.0 ± 3.0** | 43.3 ± 4.6** |
| (×10⁴ PFU/right | | | |
| lung) | | | |

Table 10 shows scores of histopathological examination of the lungs for different groups. Each value in the table is the mean and standard error of scores of histopathological examination of the lungs for the mice of the corresponding group. “*” in the table indicates the presence of significant difference from the control group (**: p<0.01).

TABLE 10
Score of histopathological examination of lung

| | Group | | |
|---|---|---|---|
| | | Group with | |
| | Control group | administration of hAM(1-52) | |
| | Dose (nmol/kg) | | |
| | 0 | 10 | 100 |
| Number of animals | 8 | 8 | 8 |
| Pneumonia score | 9.4 ± 0.6 | 8.8 ± 0.7 | 7.5 ± 0.5 |
| | Group | | |
| | | Group with administration of | |
| | Control group | [Ala-44]hAM(13-52) | |
| | Dose (nmol/kg) | | |
| | 0 | 10 | 100 |
| Number of animals | 8 | 8 | 8 |
| Pneumonia score | 9.4 ± 0.6 | 8.9 ± 0.4 | 9.0 ± 0.5 |

As demonstrated in Table 8, the gross lesion formation in the lung was significantly suppressed in the mice of the groups with administration of hAM(1-52) and the groups with administration of [Ala-44]hAM(13-52). In addition, as demonstrated in Table 9, the virus concentration in the lung significantly decreased in the mice of the groups with administration of hAM(1-52) and the groups with administration of [Ala-44]hAM(13-52).

### <Test III: Organ Protective Effect of Adrenomedullin on Influenzavirus-Infected Mice (3)>

Drug efficacy test for influenzavirus-infected mice was conducted with use of an AM derivative having a polyethylene glycol group (PEG group) to evaluate the effect of the AM derivative on organ dysfunctions in influenzavirus-infected mice.

### [III-1: Test Drug]

### (a) Active Ingredient

### 60 K PEG-AM:

An adrenomedullin derivative having a PEG group with a weight-average molecular weight of 60 kDa.

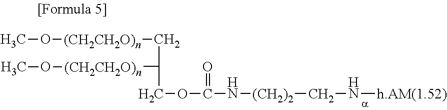

60 K PEG-AM was prepared according to WO 2017/047788.

### (b) Medium

Physiological Saline

### (c) Preparation of Specimens for Administration

The active ingredient was thawed in use, and diluted with the medium to prepare 50 or 5 nmol/mL specimen solution.

### [III-2: Test Virus Strain]

The same influenzavirus strain and host cells therefor as used in Test I were used as a virus strain and host cells therefor for use in the test. The host cells and solution of the virus to be inoculated were prepared with the same procedure as in Test I.

### [III-3: Drug Efficacy Test on Influenzavirus-Infected Mice]

Twenty-seven 5-week-old female SPF mice (BALB/c type (BALB/c Cr Slc)) were purchased (Japan SLC, Inc.). After a preliminary growing period for 6 days, the mice were grouped into a control group, a group with administration of 60 K PEG-AM at low concentration (25 nmol/kg), and a group with administration of 60 K PEG-AM at high concentration (250 nmol/kg) (eight mice in each group). During a period between the day before virus inoculation and day 4 after inoculation, the specimen solution in a liquid volume of 5 mL/kg body weight was administered via the tail vein once per day. To the mice in the control group without administration of the AM derivative, the medium was administered under the same conditions. Each mouse in each group was grown in an animal room (controlled temperature: 18 to 28° C., controlled humidity: 30 to 80% RH, 12-hour light and 12-hour dark). On the day of virus inoculation, for the mice of the control group without administration of the AM derivative and the mice of the groups with administration of the AM derivative, 0.05 mL of the solution of the virus to be inoculated per individual (1×10⁵ PFU/mouse) was dropped into the nasal cavity by using a micropipette. Growing was continued until day 5 after inoculation, as the day after the inoculation was defined as day 1 after inoculation. The same items as in Test I were evaluated with the same procedures as in Test I over the growing period.

### [III-4: Results]

Table 11 shows general condition scores for mice of different groups. Each value in the table is the mean and standard error of scores for the mice of the corresponding group. “*” in the table indicates the presence of significant difference from the control group (**: p<0.01).

TABLE 11
General condition score

| | Group | | |
|---|---|---|---|
| | | Group with administration of | |
| | Control group | 60K PEG-AM | |
| | Dose (nmol/kg) | | |
| | 0 | 25 | 250 |
| Number of animals | 8 | 8 | 8 |
| Days after | | | |
| inoculation | | | |
| 0 | 0.0 ± 0.0 | 0.0 ± 0.0 | 0.0 ± 0.0 |
| 1 | 0.0 ± 0.0 | 0.0 ± 0.0 | 0.0 ± 0.0 |
| 2 | 0.1 ± 0.1 | 0.1 ± 0.0 | 0.1 ± 0.0 |
| 3 | 0.5 ± 0.0 | 0.5 ± 0.0 | 0.5 ± 0.0 |
| 4 | 0.8 ± 0.0 | 0.7 ± 0.1 | 0.8 ± 0.0 |
| 5 | 1.0 ± 0.0 | 0.9 ± 0.1 | 1.0 ± 0.0 |

Table 12 shows body weight for mice of different groups. Each value in the table is the mean and standard error of body weight for the mice of the corresponding group.

TABLE 12
Body weight

| | Group | | |
|---|---|---|---|
| | | Group with administration of | |
| | Control group | 60K PEG-AM | |
| | Dose (nmol/kg) | | |
| | 0 | 25 | 250 |
| Number of animals | 8 | 8 | 8 |
| Days after | | | |
| inoculation | | | |
| 0 | 16.3 ± 0.4 | 16.4 ± 0.4 | 16.4 ± 0.3 |
| 1 | 16.8 ± 0.3 | 16.4 ± 0.4 | 16.1 ± 0.4 |
| 2 | 16.5 ± 0.3 | 16.3 ± 0.4 | 15.8 ± 0.5 |
| 3 | 14.8 ± 0.2 | 14.5 ± 0.4 | 14.3 ± 0.4 |
| 4 | 14.1 ± 0.3 | 13.9 ± 0.5 | 13.7 ± 0.3 |
| 5 | 13.6 ± 0.3 | 13.9 ± 0.5 | 13.3 ± 0.3 |

Table 13 shows scores of gross lesion examination of the lungs for different groups. Each value in the table is the mean and standard error of scores of gross lesion examination of the lungs for the mice of the corresponding group. “*” and “**” in the table indicate the presence of significant difference from the control group (*: p<0.05, **: p<0.01).

TABLE 13
Score of gross lesion examination of lung

| | Group | | |
|---|---|---|---|
| | | Group with administration | |
| | Control group | of 60K PEG-AM | |
| | Dose (nmol/kg) | | |
| | 0 | 25 | 250 |
| Number of animals | 8 | 8 | 8 |
| Lung | | | |
| Left lung | 3.4 ± 0.2 | 2.0 ± 0.5* | 2.4 ± 0.2** |
| Right lung | 3.4 ± 0.2 | 2.0 ± 0.5* | 1.9 ± 0.3** |

Table 14 shows virus concentrations in the lung for different groups. Each value in the table is the mean and standard error of virus concentration (×104 PFU) in the right lung for the mice of the corresponding group.

TABLE 14
Score of histopathological examination of lung

| | Group | | |
|---|---|---|---|
| | | Group with administration | |
| | Control group | of 60K PEG-AM | |
| | Dose (nmol/kg) | | |
| | 0 | 25 | 250 |
| Number of animals | 8 | 8 | 8 |
| Pneumonia score | 10.5 ± 0.7 | 8.3 ± 1.5 | 9.4 ± 0.6 |

Table 15 shows scores of histopathological examination of the lungs for different groups. Each value in the table is the mean and standard error of scores of histopathological examination of the lungs for the mice of the corresponding group.

TABLE 15
Score of histopathological examination of lung

| | Group | | |
|---|---|---|---|
| | | Group with administration | |
| | Control group | of 60K PEG-AM | |
| | Dose (nmol/kg) | | |
| | 0 | 25 | 250 |
| Number of animals | 8 | 8 | 8 |
| Pneumonia score | 10.5 ± 0.7 | 8.3 ± 1.5 | 9.4 ± 0.6 |

As demonstrated in Table 13, the gross lesion formation in the lung was significantly suppressed in the mice of the groups with administration of 60 K PEG-AM. In addition, as demonstrated in Table 14, the virus concentration in the lung significantly decreased in the mice of the groups with administration of 60 K PEG-AM.

<Test IV: Effect of Adrenomedullin on Severe Pneumonia for which Mechanical Ventilation is Needed in COVID-19 Patients>

### [IV-1: Test Drug]

### (a) Active Ingredient

A freeze-dried powder of synthetic human adrenomedullin (a peptide consisting of the amino acid sequence of SEQ ID NO: 1, amidated at the C-terminus thereof, and having a disulfide bond formed by two cysteine residues in the amino acid sequence).

### (b) Dosage Form, Content, and Characteristics:

An injection (freeze-dried product) obtained by encapsulating 500 μg of white mass or powder of the synthetic human adrenomedullin in a vial.

### (c) Preparation:

In use, 10 mL of physiological saline is injected into a vial containing the test drug, which is dissolved in such a manner that no bubble is generated. The dissolved test drug in 10 mL is taken into a syringe, to which 20 mL of physiological saline is added to dilute to a total volume of 30 mL.

### (d) Storage:

After dissolving, the resultant is stored in a sealed container under shading and refrigeration (2 to 8° C.).

A control drug (placebo), containing no active ingredient, is prepared to give the same dosage form and characteristics as the test drug. The test drug and the control drug cannot be distinguished from each other by their appearance.

### [IV-2: Subjects]

Patients who have become in need of mechanical ventilation because of COVID-19 are selected as subjects in the test. In selection of patients, patients who satisfy all the following selection criteria and do not fall within any of the following exclusion criteria are targeted.

### (a) Inclusion Criteria

(a-1) A patient who has become in need of mechanical ventilation (invasive positive-pressure ventilation) because of respiratory failure after finding COVID-19.
(a-2) A patient who is 20 years old or older and younger than 75 years old when consent to participate in the clinical trial is obtained.
(a-3) A patient for whom written consent to participate in the clinical trial has been obtained from the patient him/herself or a legally authorized representative.

### (b) Exclusion Criteria

(b-1) A patient having a past history of allergy or drug hypersensitivity that may influence the clinical trial.
(b-2) A patient who has participated or plans to participate in another clinical trial for COVID-19.
(b-3) A patient who has participated in clinical trial or clinical study with AM.
(b-4) A patient affected by hepatic dysfunction (AST or ALT is five or more times higher than the reference value).
(b-5) A patient affected by renal impairment (the serum creatinine is higher than 2.0 mg/dL).
(b-6) A patient with significant abnormal ECG, or a patient affected by clinically problematic heart disease (heart failure, myocardial infarction, angina).
(b-7) A patient whose systolic blood pressure is lower than 90 mmHg.
(b-8) A patient affected by untreated proliferative diabetic retinopathy.
(b-9) A patient affected by malignant tumor or a patient having a medical history of malignant tumor.
(b-10) A patient who is to be delivered to a hospital other than the medical institution in 72 hours.
(b-11) A female patient who is pregnant, possibly pregnant, hoping to get pregnant, or lactating.
(b-12) A patient who has been determined to be improper as a subject in the clinical trial by an investigator (subinvestigator) for any other reason.

### [IV-3: Institution]

Ten or more institutes including University of Miyazaki and National Center for Global Health and Medicine are involved.

### [IV-4: Summary of Trial]

Multi-institutional randomized double-blind trial with a placebo control is performed for patients who have become in need of mechanical ventilation because of respiratory failure after finding COVID-19. Patients selected on the basis of the criteria are assigned to a group with administration of AM and a control group. The number of patients in each group is 20.

### [IV-5: Administration Regimen]

Immediately after initiation of mechanical ventilation, administration of the test drug or the placebo is started. The test drug (15 ng active ingredient/kg body weight/min) or the placebo is intravenously administered together with physiological saline in a continuous manner for 72 hours. Thereafter, intermittent administration for 8 hours per day is continued until withdrawal from mechanical ventilation. The duration of the intermittent administration lasts up to day 10 after initiation of administration of the test drug or the placebo. The administration is discontinued for patients who have become in need of use of extracorporeal membrane oxygenation (ECMO) before day 10 after initiation of administration. The administration is immediately discontinued for patients who have been determined to be intolerant to the administration because of unstable hemodynamics.

### [IV-6: Evaluation Items]

### (a) Major Evaluation Items:

(a-1) Duration (days) with need of mechanical ventilation after administration of the test drug.

The follow-up duration is 15 days, and the duration is set to 15 days for dead cases.

Use of ECMO is regarded as use of mechanical ventilation in the calculation.

### (b) Secondary Evaluation Items:

(b-1) The clinical condition of a test subject on day 15 and day 30 after initiation of administration of the test drug.

Evaluation is performed on the basis of the following six-rank scale.
1. Without hospitalization
2. With hospitalization, without need of oxygen supply
3. With hospitalization, with need of oxygen supply
4. With hospitalization, with need of use of noninvasive artificial ventilation or a high-flow oxygen device
5. With hospitalization, with need of use of invasive mechanical artificial ventilation or ECMO
6. Death
(b-2) Change in proinflammatory cytokines and others.

The productions of IL-113, IL-6, IL-8, IL-10, TNF-α, IFN-γ, autotaxin, FDP, D-dimer, tPA, PIC, and PTX3 are evaluated.
(b-3) Incidence rates of adverse events and incidence rates of side effects.

### [IV-7: Results]

Dosing was performed for 19 patients by June 2021. Currently, which patients the test drug or the placebo was administered to is unknown. Table 16 shows a list of cases with complete data until day 30 after initiation of administration of the test drug or the placebo. Three patients died, and these cases are not included in Table 16. Numerical values shown in the column Clinical condition are the above-described evaluation scales for clinical condition. For numerical values shown in the column Clinical severity, higher values indicate being more severe. For numerical values shown in the column Production of proinflammatory cytokine (CRP, D-dimer, IL-6, and PTX-3), higher values indicate the presence of more intense inflammation.

TABLE 16

| | | | Clinical severity | | | | | | | | |
|---|---|---|---|---|---|---|---|---|---|---|---|
| | | | (before) | | Production of proinflammatory cytokine | | | | Duration of | | |
| | | | | APACHE | (before) | | | | mechanical | Clinical condition | |
| | Age | Sex | SOFA | II | CRP | D-dimer | IL-6 | PTX3 | ventilation | Day 15 | Day 30 |
| 1 | 50s | male | 2 | 4 | 5.6 | 2.37 | 11.2 | 35.13 | 9 days | 3 | 2 |
| 2 | 40s | female | 3 | 7 | 6.94 | 1.46 | 49.0 | 57.39 | 11 days | 4 | 3 |
| 3 | 60s | male | 4 | 12 | 6.39 | 0.59 | 15.9 | 29.53 | 5 days | 3 | 1 |
| 4 | 40s | female | 4 | 9 | 31.28 | 1.07 | 30.3 | 11,22 | 11 days | 3 | 1 |
| | | | | | | | | | (ECMO: | | |
| | | | | | | | | | 10 days) | | |
| 5 | 60s | male | 4 | 9 | 0.68 | 7.83 | 1090 | 57.29 | 5 days | 3 | 3 |
| 6 | 70s | male | 3 | 6 | 11.65 | 30.00 | 16.4 | 125.40 | 4 days | 3 | 3 |
| 7 | 60s | male | 6 | 10 | 6.48 | 2.22 | 115 | 15.09 | 6 days | 3 | 3 |
| 8 | 60s | male | 7 | 9 | 5.52 | 0.55 | 13.2 | 50.83 | 15 days | 5 | 5 |
| | | | | | | | | | (ECMO: | | |
| | | | | | | | | | 9 days) | | |
| 9 | 40s | male | 4 | 3 | 15.3 | 1.04 | 6.0 | 13.77 | 4 days | 1 | 1 |
| 10 | 50s | male | 8 | 18 | 8.96 | 1.15 | 58.7 | 77.26 | 5 days | 2 | 1 |
| 11 | 70s | female | 9 | 33 | 3.94 | 0.65 | 126 | 98.98 | 15 days | 5 | 5 |
| 12 | 50s | male | 7 | 15 | 28.6 | 2.11 | 138 | 55.69 | 7 days | 3 | 3 |

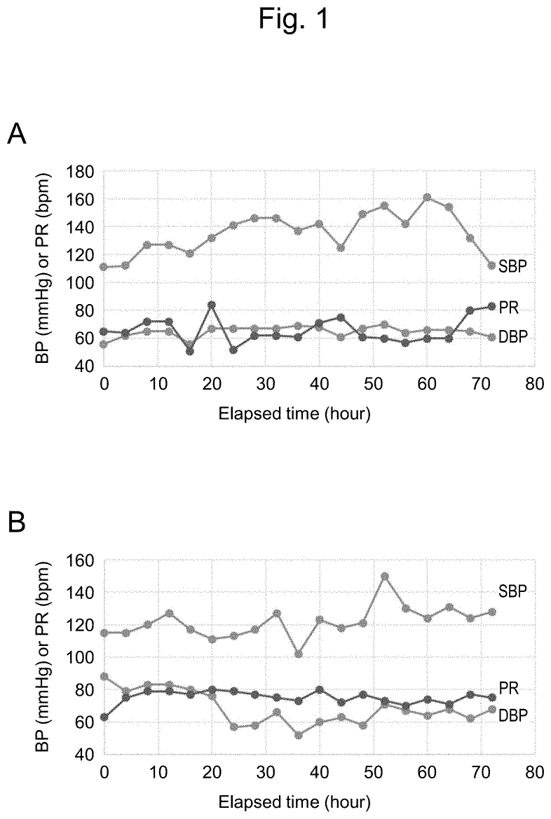

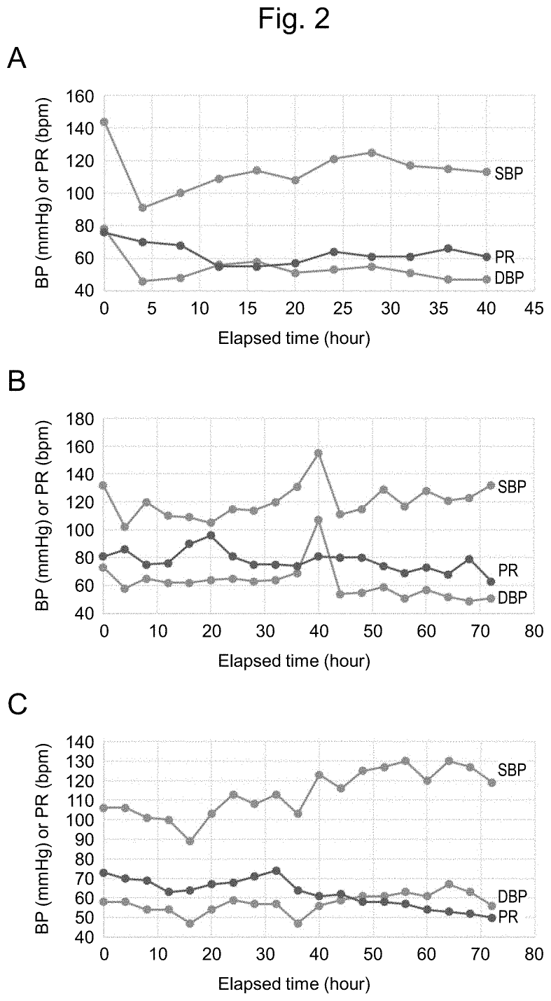

As demonstrated in Table 16, in the distribution of duration of mechanical ventilation, there were a group with short duration of mechanical ventilation of around 5 days and a group with long-term mechanical ventilation for 9 to 15 days. FIG. 1 shows time course of blood pressure for cases 2 and 8 in the group with long-term mechanical ventilation for 9 to 15 days, wherein the cases showed relatively stable blood pressure during continuous administration for 72 hours. FIG. 2 shows time course of blood pressure for cases 3, 5, and 10 in the group with short duration of mechanical ventilation of around 5 days, wherein the cases showed relatively stable blood pressure during continuous administration for 72 hours. In FIG. 1, A and B show the results for case 2 and case 8, respectively. In FIGS. 2, A, B, and C show the results for case 3, case 5, and case 10, respectively. In each figure, BP, DBP, SBP, and PR indicate blood pressure, diastolic blood pressure, systolic blood pressure, and pulse rates, respectively. The abscissa represents elapsed time (hour), and the ordinate represents BP (mmHg) or PR (bpm).

As demonstrated in FIG. 1, significant reduction in blood pressure was not observed for the group with long-term mechanical ventilation after administration of the test drug or the placebo. As demonstrated in FIG. 2, by contrast, reduction in blood pressure was observed for the group with short-term mechanical ventilation after administration of the test drug or the placebo, and increase in blood pressure (inferred as pathological recovery) was observed in the latter half of the duration.

As described above, the present trial is randomized double-blind trial, and it is currently difficult to show a definite conclusion. However, reduction in blood pressure was observed for the cases with short-term mechanical ventilation in the early stage of administration (FIG. 2). This is inferred to be the effect of AM. In particular, case 10 (FIG. 2C) succeeded in early withdrawal from mechanical ventilation in spite of the high clinical severity and the high inflammatory reaction (i.e., high production of cytokine), and the subsequent progress was very good as well. These findings probably suggest the efficacy of AM in clinical situations. In addition, no adverse event directly caused by AM has been reported so far in patients subjected to dosing. Probably, this ensures the safety of AM.

On the basis of the administration regimen of the present trial, AM is administered to a COVID-19 patient affected by severe pneumonia for which mechanical ventilation is needed, and the drug concentration is continuously maintained. Administration of AM at night, in which reduction in blood pressure is expected, has been conventionally avoided from the viewpoint of management of blood pressure. However, the doses of AM in the administration regimen of the present trial is such amounts that reduction in blood pressure is not directly caused. Therefore, dosage and administration that cannot be achieved by conventional clinical administration of AM can be achieved by administering AM on the basis of the administration regimen of the present trial, with the quick exertion of drug efficacy of AM and the safety ensured in combination.

Symptoms or disorders in viral pneumonia patients, in particular, COVID-19 patients, in particular, organ dysfunctions can be significantly prevented or treated by administering AM on the basis of the administration regimen of the present trial. This also results in improved prognosis in viral pneumonia patients, in particular, COVID-19 patients.

The invention is not limited to Examples shown above, and includes various modifications. For example, Examples shown above are detail description for easy-to-understand explanation of the invention, and not necessarily limited to the configuration including all the components described. In addition, some of the components in each Example may be supplemented with other components, deleted, and/or substituted.

All publications, patents, and patent applications cited herein are incorporated herein by reference in their entirety.

1-20. (canceled)

21. A method for preventing or treating a symptom or disorder due to a viral infection or a sequela after cure of a viral infection, which comprises administering an effective amount of the adrenomedullin or a derivative thereof, to a subject affected by the viral infection or a subject affected by the sequela after cure of the viral infection.

22. The method according to claim 21 for preventing or treating a symptom or disorder in a subject affected by a viral infection.

23. The method according to claim 21, wherein the effective amount of the adrenomedullin or a derivative thereof is administered as a pharmaceutical composition comprising the adrenomedullin or a derivative thereof and one or more pharmaceutically acceptable carriers.

24. The method according to claim 21, wherein the adrenomedullin or a derivative thereof is a peptide or compound or a pharmaceutically acceptable salt thereof, or a pharmaceutically acceptable solvate thereof, the peptide or compound being selected from the group consisting of:
(i) a peptide consisting of an amino acid sequence of adrenomedullin,
(ii) a peptide consisting of an amino acid sequence of adrenomedullin and having a disulfide bond formed by two cysteine residues in the amino acid sequence,
(iii) a peptide wherein the disulfide bond of the peptide of (ii) is substituted with an ethylene group,
(iv) a peptide wherein 1 to 15 amino acid residues of any of the peptides of (i) to (iii) are deleted, substituted, or added,
(v) a peptide wherein any of the peptides of (i) to (iv) is amidated at the C-terminus thereof,
(vi) a peptide wherein any of the peptides of (i) to (iv) has a glycine residue added to the C-terminus thereof, and
(vii) a compound represented by formula (A-I):
A-Lₙ-B (A-I)
wherein
A is a modifying group,
L is a divalent linking group,
n is an integer of 0 or 1, and
B is a peptide group having a structure formed by eliminating one hydrogen atom from the N-terminal amino group of any of the peptides of (i) to (vi),
provided that modifying group A is bound to the N-terminal amino group of peptide group B with or without intervening linking group L.

25. The method according to claim 24, wherein the adrenomedullin or a derivative thereof is a peptide or compound or a pharmaceutically acceptable salt thereof, or a pharmaceutically acceptable solvate thereof, the peptide or compound being selected from the group consisting of:
(i) a peptide consisting of an amino acid sequence of adrenomedullin,
(ii) a peptide consisting of an amino acid sequence of adrenomedullin and having a disulfide bond formed by two cysteine residues in the amino acid sequence,
(v) a peptide wherein the peptide of (i) or (ii) is amidated at the C-terminus thereof,
(vi) a peptide wherein the peptide of (i) or (ii) has a glycine residue added to the C-terminus thereof, and
(vii) a compound represented by formula (A-I):
A-Lₙ-B (A-I)
wherein
A is a modifying group,
L is a divalent linking group,
n is an integer of 0 or 1, and
B is a peptide group having a structure formed by eliminating one hydrogen atom from the N-terminal amino group of any of the peptides of (i), (ii), (v), and (vi),
provided that modifying group A is bound to the N-terminal amino group of peptide group B with or without intervening linking group L.

26. The method according to claim 21, wherein the adrenomedullin or a derivative thereof is a peptide or compound or a pharmaceutically acceptable salt thereof, or a pharmaceutically acceptable solvate thereof, the peptide or compound being selected from the group consisting of:
(a) a peptide consisting of an amino acid sequence of SEQ ID NO: 1, or a peptide consisting of an amino acid sequence of SEQ ID NO: 1, and having a disulfide bond formed by a cysteine residue at position 16 and a cysteine residue at position 21;
(b) a peptide consisting of an amino acid sequence of SEQ ID NO: 4, or a peptide consisting of an amino acid sequence of SEQ ID NO: 4, and having a disulfide bond formed by a cysteine residue at position 16 and a cysteine residue at position 21;
(c) a peptide consisting of an amino acid sequence of SEQ ID NO: 6, or a peptide consisting of an amino acid sequence of SEQ ID NO: 6, and having a disulfide bond formed by a cysteine residue at position 16 and a cysteine residue at position 21;
(d) a peptide consisting of an amino acid sequence of SEQ ID NO: 8, or a peptide consisting of an amino acid sequence of SEQ ID NO: 8, and having a disulfide bond formed by a cysteine residue at position 16 and a cysteine residue at position 21;
(e) a peptide consisting of an amino acid sequence of SEQ ID NO: 10, or a peptide consisting of an amino acid sequence of SEQ ID NO: 10, and having a disulfide bond formed by a cysteine residue at position 14 and a cysteine residue at position 19;
(f) a peptide consisting of an amino acid sequence of SEQ ID NO: 12, or a peptide consisting of an amino acid sequence of SEQ ID NO: 12, and having a disulfide bond formed by a cysteine residue at position 14 and a cysteine residue at position 19;
(g) a peptide wherein the disulfide bond of any of the peptides of (a) to (f) is substituted with an ethylene group;
(h) a peptide wherein 1 to 15 amino acid residues of any of the peptides of (a) to (g) are deleted, substituted, or added;
(i) a peptide wherein any of the peptides of (a) to (h) is amidated at the C-terminus thereof;
(j) a peptide wherein any of the peptides of (a) to (h) has a glycine residue added to the C-terminus thereof; and
(k) a compound represented by formula (A-I):
A-Lₙ-B (A-I)
wherein
A is a modifying group,
L is a divalent linking group,
n is an integer of 0 or 1, and
B is a peptide group having a structure formed by eliminating one hydrogen atom from the N-terminal amino group of any of the peptides of (a) to (j),
provided that modifying group A is bound to the N-terminal amino group of peptide group B with or without intervening linking group L.

27. The method according to claim 26, wherein the adrenomedullin or a derivative thereof is a peptide or compound or a pharmaceutically acceptable salt thereof, or a pharmaceutically acceptable solvate thereof, the peptide or compound being selected from the group consisting of:
(a) a peptide consisting of an amino acid sequence of SEQ ID NO: 1, or a peptide consisting of an amino acid sequence of SEQ ID NO: 1, and having a disulfide bond formed by a cysteine residue at position 16 and a cysteine residue at position 21;
(b) a peptide consisting of an amino acid sequence of SEQ ID NO: 4, or a peptide consisting of an amino acid sequence of SEQ ID NO: 4, and having a disulfide bond formed by a cysteine residue at position 16 and a cysteine residue at position 21;
(c) a peptide consisting of an amino acid sequence of SEQ ID NO: 6, or a peptide consisting of an amino acid sequence of SEQ ID NO: 6, and having a disulfide bond formed by a cysteine residue at position 16 and a cysteine residue at position 21;
(d) a peptide consisting of an amino acid sequence of SEQ ID NO: 8, or a peptide consisting of an amino acid sequence of SEQ ID NO: 8, and having a disulfide bond formed by a cysteine residue at position 16 and a cysteine residue at position 21;
(e) a peptide consisting of an amino acid sequence of SEQ ID NO: 10, or a peptide consisting of an amino acid sequence of SEQ ID NO: 10, and having a disulfide bond formed by a cysteine residue at position 14 and a cysteine residue at position 19;
(f) a peptide consisting of an amino acid sequence of SEQ ID NO: 12, or a peptide consisting of an amino acid sequence of SEQ ID NO: 12, and having a disulfide bond formed by a cysteine residue at position 14 and a cysteine residue at position 19;
(i) a peptide wherein any of the peptide of (a) to (f) is amidated at the C-terminus thereof;
(j) a peptide wherein any of the peptides of (a) to (f) has a glycine residue added to the C-terminus thereof; and
(k) a compound represented by formula (A-I):
A-Lₙ-B (A-I)
wherein
A is a modifying group,
L is a divalent linking group,
n is an integer of 0 or 1, and
B is a peptide group having a structure formed by eliminating one hydrogen atom from the N-terminal amino group of any of the peptides of (a) to (f), (i), and (j),
provided that modifying group A is bound to the N-terminal amino group of peptide group B with or without intervening linking group L.

28. The method according to claim 21, wherein the peptide of (iv) in the adrenomedullin or a derivative thereof is a peptide wherein one to three amino acid residues of a peptide consisting of an amino acid sequence of SEQ ID NO: 14 are substituted or deleted.

29. The method according to claim 24, wherein modifying group A in formula (A-I) is a modifying group selected from the group consisting of an organic group comprising a C₄-C₃₀ alkyl group, a C₄-C₃₀ alkenyl group or a polyethylene glycol group, an Fc region of an immunoglobulin, and serum albumin.

30. The method according to claim 24, wherein, in formula (A-I),
A is an organic group comprising a polyethylene glycol group, and
L is a divalent linking group represented by formula (z): 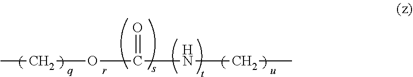
wherein
q and u are the same or different, and each represent an integer of 0 to 6,
r, s, and t are the same or different, and each represent an integer of 0 or 1, and
n is the integer 1.

31. The method according to claim 30, wherein the organic group comprising a polyethylene glycol group is a group represented by any of formulas (β) to (δ): 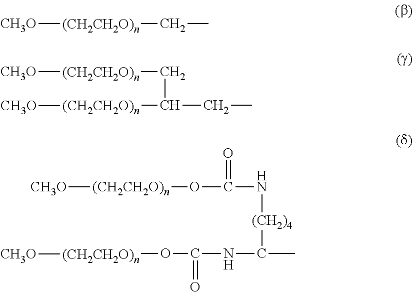
and,
having a weight-average molecular weight ranging from 20 to 80 kDa.

32. The method according to claim 21, wherein the adrenomedullin or a derivative thereof is adrenomedullin or a derivative thereof with adrenomedullin activity.

33. The method according to claim 21, wherein the viral infection is viral pneumonia, viral myocarditis, viral encephalitis, viral hemorrhagic fever, viral nephropathy, viral gastroenteritis, viral vasculitis, viral stomatitis, viral keratitis, or viral neuritis.

34. The method according to claim 21, wherein the viral infection in the subject affected by the viral infection is caused by one or more viruses selected from the group consisting of novel coronavirus (SARS-CoV-2), severe acute respiratory syndrome (SARS) coronavirus, Middle East respiratory syndrome (MERS) coronavirus, conventional human coronavirus (229E, NL63, OC43, and HKU1), influenzavirus, dengue virus, RS virus, adenovirus, varicella-zoster virus, herpes simplex virus, measles virus, parainfluenza virus, enterovirus, rhinovirus, and human metapneumovirus.

35. The method according to claim 21, wherein the sequela remaining after cure of the viral infection is dyspnea or shortness of breath remaining after cure of viral pneumonia.

36. The method according to claim 21, wherein the adrenomedullin or a derivative thereof as an active ingredient is applied through intravenous administration in a continuous manner.

37. The method according to claim 36, wherein the intravenous administration is performed in a continuous manner at a rate ranging from 1.0 to 100.0 ng active ingredient/kg body weight/min.

38. The method according to claim 36, wherein the intravenous administration is performed with continuous administration for 72 hours followed by intermittent administration for 8 hours per day.

39. The method according to claim 36, wherein the intravenous administration is performed for 3 to 10 days after initiation of administration.

40. The method according to claim 36, wherein the intravenous administration is performed on the basis of an administration regimen such that the intravenous administration is performed with continuous administration for 72 hours followed by intermittent administration for 8 hours per day for 3 to 10 days after initiation of administration.


SEQUENCE LISTING (28 sequences)

SEQ ID NO: 1 - protein, 52 aa, Homo sapiens
YRQSMNNFQGLRSFGCRFGTCTVQKLAHQIYQFTDKDKDNVAPRSKISPQGY

SEQ ID NO: 2 - DNA, 1449 bp, Homo sapiens
  CDS (157)..(711)
ctggatagaacagctcaagccttgccacttcgggcttctcactgcagctgggcttggacttcggagttttgccattgccagtgggacgtctgagactttctccttcaagtacttggcagatcactctcttagcagggtctgcgcttcgcagccgggatgaagctggtttccgtcgccctgatgtacctgggttcgctcgccttcctaggcgctgacaccgctcggttggatgtcgcgtcggagtttcgaaagaagtggaataagtgggctctgagtcgtgggaagagggaactgcggatgtccagcagctaccccaccgggctcgctgacgtgaaggccgggcctgcccagacccttattcggccccaggacatgaagggtgcctctcgaagccccgaagacagcagtccggatgccgcccgcatccgagtcaagcgctaccgccagagcatgaacaacttccagggcctccggagctttggctgccgcttcgggacgtgcacggtgcagaagctggcacaccagatctaccagttcacagataaggacaaggacaacgtcgcccccaggagcaagatcagcccccagggctacggccgccggcgccggcgctccctgcccgaggccggcccgggtcggactctggtgtcttctaagccacaagcacacggggctccagcccccccgagtggaagtgctccccactttctttaggatttaggcgcccatggtacaaggaatagtcgcgcaagcatcccgctggtgcctcccgggacgaaggacttcccgagcggtgtggggaccgggctctgacagccctgcggagaccctgagtccgggaggcaccgtccggcggcgagctctggctttgcaagggcccctccttctgggggcttcgcttccttagccttgctcaggtgcaagtgccccagggggcggggtgcagaagaatccgagtgtttgccaggcttaaggagaggagaaactgagaaatgaatgctgagacccccggagcaggggtctgagccacagccgtgctcgcccacaaactgatttctcacggcgtgtcaccccaccagggcgcaagcctcactattacttgaactttccaaaacctaaagaggaaaagtgcaatgcgtgttgtacatacagaggtaactatcaatatttaagtttgttgctgtcaagattttttttgtaacttcaaatatagagatatttttgtacgttatatattgtattaagggcattttaaaagcaattatattgtcctcccctattttaagacgtgaatgtctcagcgaggtgtaaagttgttcgccgcgtggaatgtgagtgtgtttgtgtgcatgaaagagaaagactgattacctcctgtgtggaagaaggaaacaccgagtctctgtataatctatttacataaaatgggtgatatgcgaacagcaaacc

SEQ ID NO: 3 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 4 - protein, 52 aa, Sus scrofa
YRQSMNNFQGLRSFGCRFGTCTVQKLAHQIYQFTDKDKDGVAPRSKISPQGY

SEQ ID NO: 5 - DNA, 1493 bp, Sus scrofa
gcggaacagctcgagccttgccacctctagtttcttaccacagcttggacgtcggggttttgccactgccagagggacgtctcagacttcatcttcccaaatcttggcagatcacccccttagcagggtctgcacatctcagccgggatgaagctggttcccgtagccctcatgtacctgggctcgctcgccttcctgggcgctgacacagctcggctcgacgtggcggcagagttccgaaagaaatggaataagtgggctctaagtcgtggaaaaagagaacttcggctgtccagcagctaccccaccgggatcgccgacttgaaggccgggcctgcccagactgtcattcggccccaggatgtgaagggctcctctcgcagcccccaggccagcattccggatgcagcccgcatccgagtcaagcgctaccgccagagtatgaacaacttccagggcctgcggagcttcggctgtcgctttgggacgtgcaccgtgcagaagctggcgcaccagatctaccagttcacggacaaagacaaggacggcgtcgccccccggagcaagatcagcccccagggctacggccgccggcgccgacgctctctgcccgaagccagcctgggccggactctgaggtcccaggagccacaggcgcacggggccccggcctccccggcgcatcaagtgctcgccactctctttaggatttaggcgcctactgtggcagcagcgaacagtcgcgcatgcatcatgccggcgcttcctggggcggggggcttcccggagccgagcccctcagcggctggggcccgggcagagacagcattgagagaccgagagtccgggaggcacagaccagcggcgagccctgcattttcaggaacccgtcctgcttggaggcagtgttctcttcggcttaatccagcccgggtccccgggtgggggtggagggtgcagaggaatccaaaggagtgtcatctgccaggctcacggagaggagaaactgcgaagtaaatgcttagacccccaggggcaagggtctgagccactgccgtgccgcccacaaactgatttctgaaggggaataaccccaacagggcgcaagcctcactattacttgaactttccaaaacctagagaggaaaagtgcaatgtatgttgtatataaagaggtaactatcaatatttaagtttgttgctgtcaagatttttttttgtaacttcaaatatagagatatttttgtacgttatatattgtattaagggcattttaaaacaattgtattgttcccctcccctctattttaatatgtgaatgtctcagcgaggtgtaacattgtttgctgcgcgaaatgtgagagtgtgtgtgtgtgtgtgcgtgaaagagagtctggatgcctcttggggaagaagaaaacaccatatctgtataatctatttacataaaatgggtgatatgcgaagtagcaaaccaataaactgtctcaatg

SEQ ID NO: 6 - protein, 52 aa, Canis familiaris
YRQSMNNFQGPRSFGCRFGTCTVQKLAHQIYQFTDKDKDGVAPRSKISPQGY

SEQ ID NO: 7 - DNA, 1432 bp, Canis familiaris
ggttttgccagcaccagagcgacgtctcagaccttctcctcccggatcttggcagatcaccccctcagcagggtctgcgcatcgccgccagcatgaagctggttcccgtcgccctcttatacctgggctccctcgccttcttgggcgcggacaccgcacggctagacgtggcgtcagagttccgaaagaagtggaataaatgggctgtaagtcgtggaaagagggaacttcgagtgtccagcagctatcccaccgggctcgctgaagtgaaggccgggccggcccagactcttattcggacccaggacgtgaagggcgcctctcgcaacccccagaccagcggtccggacgccgcccgcatccgagtcaaacgctaccgccagagtatgaacaatttccagggcccgcggagcttcggctgccgcttcggaacgtgcacggtgcagaaactggcgcaccagatctaccagttcacagacaaggacaaggacggcgtcgcccccaggagcaagattagccctcagggctacggccgccggcgccggcgctccctgcccgagcccggccttcgccggactctgttgttcccggagccacggccaggcggggctccggccccccgggcgcatcaggtgctcgccaacctccttaagatgtaggcgcctgtggcagcagcgaactggcgcgcgtgtgcatcccgctggcttccccctgggcggagggcttccccgagccgagcccctctgccgatggaagtcgggcagagaccgggattccgggaggcaccgtcccgcggccagccctggctttgcgcgagccccttctcctcggaggcacggatccctctgtcccaagccggcccaggtgtcccgtggggggcagaggaatgcaagggaggcctgccaggctcacggagaggattaactgagaattaaatgagaattaaatgcttgagaccctcccccctccccccccagggacaggggtctgagtcactgccgtgcctgcccacaaactgatttctcacggggtgtcaccccaccggggcgcaagcctcactattacttgaactttccaaaacctagagaggaaaagtgcaatgcgtgttgtatatacagaggtaactatcaatatttaagttcgttgctgtcagaagattttttttgtaacttcaaatatagagatatttttgtacgttatatattgtattaagggcatttaaaaaccattgcattgtccccctccccacttattttaatacgtgaatgtctcagcgaggtgtaacgttgtttttgctgcagagtgtgtgagtgtgcgtgagagacttattacctcttgtggaagaaggaacaccgtgtctctgcattatctatttacataaaatgggtgatatgcgaaaatagcaaatcaataataaacggtctcgatgctg

SEQ ID NO: 8 - protein, 52 aa, Bos taurus
YRQSLNNFQGLRSFGCRFGTCTVQKLAHQIYHFTDKDKDGSAPRSKISPQGY

SEQ ID NO: 9 - DNA, 1439 bp, Bos taurus
cgggaaacagctcgaaccttctcacttttggcttctcactgcagcttcgacgtcggggttttgccactgccagaacgccgtctcagacttaatactccaaagaattttggcagatcaccccctcagcagggtctgcgcatcgccgccgggatgaagctggttcccgtcgccctcctgtacctggggtcgctcgccttcctaggcgtggacacggcacggctcgacgtggcggcagagttccgaaagaaatggaataagtgggctctaagtcgtggaaaaagagaacttcgcgagtccagtagctaccccaccgggctcgccgacgtgaaggccgggcctgtccagactcttattcggccccaggatgtaaagggcgcctctcgaagccctcaggccagcagtcctgacgcagcccgcatccgagtcaagcgctaccgccagagtttgaacaacttccagggcctgcggagcttcggttgtcgcttcgggacatgcacggtgcagaagttggcgcatcagatctaccatttcacggacaaggacaaggacggatccgcccccaggagcaagatcagcccccagggctacggccgtcggcgccgacgttcactgcctgaggccggcttgggtcggactctattacagcctccagagccaaagctgcgaggggccccggactcccgggtgcatcaagtacttgccaccctcaggatttaggcgcctgggcagcagcgaacagtcgcgcacgcatctcgccggcacctcttcgggcgggagggcttccgcgagccgagcccctcactcagcctatgggcccgggctgagaacagccctgagagaccgagagtccaggaggcaccgtccggcagccagcgagcactggctttgcaggaacccgtcctcctcggaggggaggcagtgttctcttcactctaattggggccaggtgcagtttctcctctccgtgagcctggcagacgctcacggagaggagaaactgcgaaataaatgatgagaccctcaggggcaagggtctgagccactgccgtgcccgcccacaaactgattcctgatgggggtgtcaccccaccggggtgcaagcctcactattacttgaactttccgaaacctagagaggaaaagtgcaatgagtgttgtatatacagagataattatcaatatttaaatttgttgttgtcaagattttttttgtaacttcaaatatagagatatttttgtacgttatatattgtattaagggcattttaaagcaattgtattgttcccctcccctctattttaataagtgaatgtctcagcgagatgcaacgttgtttgctgcgtggaatgtgagagtgtgtgcgtgaaagagatgagttgcctcttgtggaagaagaaaacaccgtgtctgtataatctatttacataaagtgggccgg

SEQ ID NO: 10 - protein, 50 aa, Rattus norvegicus
YRQSMNQGSRSTGCRFGTCTMQKLAHQIYQFTDKDKDGMAPRNKISPQGY

SEQ ID NO: 11 - DNA, 1376 bp, Rattus norvegicus
tccagcctttaccgctcctggtttctcggcttctcatcgcagtcagtcttggactttgcgggttttgccgctgtcagaaggacgtctcggactttctgcttcaagtgcttgacaactcaccctttcagcagggtatcggagcatcgctacagaatgaagctggtttccatcgccctgatgttattgggttcgctcgccgttctcggcgcggacaccgcacggctcgacacttcctcgcagttccgaaagaagtggaataagtgggcgctaagtcgtgggaagagggaactacaagcgtccagcagctaccctacggggctcgttgatgagaagacagtcccgacccagactcttgggctccaggacaagcagagcacgtctagcaccccacaagccagcactcagagcacagcccacattcgagtcaaacgctaccgccagagcatgaaccaggggtcccgcagcactggatgccgctttgggacctgcacaatgcagaaactggctcaccagatctaccagtttacagacaaagacaaggacggcatggcccccagaaacaagatcagccctcaaggctatggccgccggcgccggcgttccctgccagaggtcctccgagcccggactgtggagtcctcccaggagcagacacactcagctccagcctccccggcgcaccaagacatctccagagtctctaggttataggtgcgggtggcagcattgaacagtcgggcgagtatcccattggcgcctgcggaatcagagagcttcgcaccctgagcggactgagacaatcttgcagagatctgcctggctgcccctaggggaggcagaggaacccaagatcaagccaggctcacgtcagaaaccgagaattacaggctgatactctctccgggcaggggtctgagccactgccttgcccgctcataaactggttttctcacggggcatacggctcattacttacttgaactttccaaaacctagcgaggaaaagtgcaatgcttgttatacagccaaaggtaactatcatatttaagtttgttgatgtcaagaggtttttttttttgtaacttcaaatatatagaaatatttttgtacgttatatattgtattaagggcattttaaagcgattatattgtcaccttcccctattttaagaagtgaatgtctcagcaaggtgtaaggttgtttggttccgtgtgtgtgtgtgtgtgtgtgtgtgtgtgtgtgtgtgtgtgtgtgtaaggtggagagcgcctgattaccgcctgtggatgaagaaaaaacattgtgtcttctataatctatttacataaaatatgtgatctgggaaaaagcaaaccaataaactgtctcaatgctg

SEQ ID NO: 12 - protein, 50 aa, Mus musculus
YRQSMNQGSRSNGCRFGTCTFQKLAHQIYQLTDKDKDGMAPRNKISPQGY

SEQ ID NO: 13 - DNA, 1381 bp, Mus musculus
cttggtgacactagacagagcaactccagcgttaccgctcccgctcctggtttctcggcttctcatcgcagtcaatcttggactttggggttttgctactgtcagaaggacttctttctgcttcaagtgcttgacaacgcacccctttatcagggtatcagagcatcgccacagaatgaagctggtttccatcaccctgatgttattgggttcactcgctttcctaggcgcggacactgcagggccagatactccttcgcagttccgaaagaagtggaataagtgggcgctaagtcgtgggaagagggaactacaagcatccagcagctaccctacgggactcgctgatgagacgacagttcctacccagactcttgatccattcctggacgagcagaacacaactggccccctacaagccagcaatcagagcgaagcccacattcgtgtcaaacgctaccgccagagcatgaaccagggttcccgcagcaatggatgccgcttcgggacctgcacatttcagaaattggcccaccagatctaccagctaacagacaaagacaaggacggcatggctcccagaaacaagatcagccctcaaggctatggccgccggcgccggcgttccctgctggaggtcctccggtcccggactgtggagtcctcccaggagcagacacacacagccccaggcccctgggcgcacatctccagactctttaggatataggtgcgggtgacagcattgaacagtcgggcgagtatcccgttggcgcctgcggaatcagagaacttcgcaccggggcggactgagacaatcctgcagagatctgcctggctgcccctaggggaggcagaggaacccaagaccaagccaggctcatgccagaaaccgagacttacaggctgatactctccgggcaggggtctgagccactgccttgcccgctcataaactggtttctcacggggcataagcctcattactacttgaactttccaaaacctagcgaggaacgtgcaatgcttgttgtccagccaaaggtaactatagtatttaagtttgttgctgtcaaggtttttttttttgtaacttcaaatatatagagatatttttgtacgttatatattgtattaagggcattttaaagtgattatattgtcaccttcccctattttaagacgtgaatgtctcagcaaggtgtaaggttgtttggttccgtgtgtgtgtgtgtgtgtgtgtgtgtgtgtgtgtgtgtgtaaggtggagagcgcctgattatcgcctgtggatgaagaaaaaacattgtgtttcctataatctatttacataaaatatgtgatctgggaaaaagcaaaccaataaactgtctcaatgctg

SEQ ID NO: 14 - protein, 40 aa, Artificial
  Synthetic
SFGCRFGTCTVQKLAHQIYQFTDKDKDNVAPRSKISPQGY

SEQ ID NO: 15 - protein, 40 aa, Artificial
  Synthetic
  misc_feature (32)..(32): Xaa is D-Arg
SFGCRFGTCTVQKLAHQIYQFTDKDKDNVAPXSKISPQGY

SEQ ID NO: 16 - protein, 40 aa, Artificial
  Synthetic
SFGCRFGTCTVQKLAHQIYQFTDKDKDNVAPKSKISPQGY

SEQ ID NO: 17 - protein, 40 aa, Artificial
  Synthetic
SFGCRFGTCTVQKLAHQIYQFTDKDKDNVAPASKISPQGY

SEQ ID NO: 18 - protein, 40 aa, Artificial
  Synthetic
SFGCRFGTCTVQKLAHQIYQFTDKDKDNVAPGSKISPQGY

SEQ ID NO: 19 - protein, 39 aa, Artificial
  Synthetic
SFGCRFGTCTVQKLAHQIYQFTDKDKDNVAPSKISPQGY

SEQ ID NO: 20 - protein, 40 aa, Artificial
  Synthetic
SFGCRFGTCTVQKLAHQIYQFTDKDKDNVAPDSKISPQGY

SEQ ID NO: 21 - protein, 40 aa, Artificial
  Synthetic
SFGCRFGTCTVQKLAHQIYQFTDKDKDNVAPFSKISPQGY

SEQ ID NO: 22 - protein, 40 aa, Artificial
  Synthetic
SFGCRFGTCTVQKLAHQIYQFTDKDKDNVAPRPKISPQGY

SEQ ID NO: 23 - protein, 4 aa, Artificial
  linker
GGGS

SEQ ID NO: 24 - protein, 4 aa, Artificial
  linker
GGGK

SEQ ID NO: 25 - protein, 5 aa, Artificial
  linker
GGGGS

SEQ ID NO: 26 - protein, 5 aa, Artificial
  linker
GGGGK

SEQ ID NO: 27 - protein, 15 aa, Artificial
  linker
GGGGSGGGGSGGGGS

SEQ ID NO: 28 - protein, 15 aa, Artificial
  linker
GGGGSGGGGSGGGGK